SEQUENCE LISTING (18 sequences)

SEQ ID NO: 1 - DNA, 357 bp, synthetic construct
  misc_feature 1..357: note = synthetic
  source 1..357: mol_type = other DNA
caggtgcagctggtgcagtctggggctgaggtgaagaagcctggggcctcagtgaaggtctcctgcaaggtttccggattcaccctcactgaattatccattcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgaccagcctgagatcggaagacacggccgtgtattactgttcaacgatttttggagtggttaccaactttgacaactggggccagggaaccctggtcaccgtctcctca

SEQ ID NO: 2 - protein, 119 aa, synthetic construct [sequence omitted]

SEQ ID NO: 3 - DNA, 24 bp, synthetic construct
  misc_feature 1..24: note = synthetic
  source 1..24: mol_type = other DNA
ggattcaccctcactgaattatcc

SEQ ID NO: 4 - protein, 8 aa, synthetic construct
  REGION 1..8: note = synthetic
GFTLTELS

SEQ ID NO: 5 - DNA, 24 bp, synthetic construct
  misc_feature 1..24: note = synthetic
  source 1..24: mol_type = other DNA
tttgatcctgaagatggtgaaaca

SEQ ID NO: 6 - protein, 8 aa, synthetic construct
  REGION 1..8: note = synthetic
FDPEDGET

SEQ ID NO: 7 - DNA, 36 bp, synthetic construct
  misc_feature 1..36: note = synthetic
  source 1..36: mol_type = other DNA
tcaacgatttttggagtggttaccaactttgacaac

SEQ ID NO: 8 - protein, 12 aa, synthetic construct
  REGION 1..12: note = synthetic
STIFGVVTNFDN

SEQ ID NO: 9 - DNA, 324 bp, synthetic construct
  misc_feature 1..324: note = synthetic
  source 1..324: mol_type = other DNA
gacatccagatgacccagtctccatcctccctgtctgcatctgcaggagacagagtcaccatcacttgccgggcaagtcaggccattagaaatgatttaggctggtatcagcagaaaccagggaaagcccctaagcgcctgatctatgctgcattcaatttgcaaagtggggtcccatcaagattcagcggcagtggatctgggacagaattcactctcacaatcagtagcctgcagcctgaagatcttgcaagttattactgtcaacagtataatagatacccgtggacgttcggccaagggaccaaggtggaaatcaaacga

SEQ ID NO: 10 - protein, 108 aa, synthetic construct
  REGION 1..108: note = synthetic
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKVEIKR

SEQ ID NO: 11 - DNA, 18 bp, synthetic construct
  misc_feature 1..18: note = synthetic
  source 1..18: mol_type = other DNA
caggccattagaaatgat

SEQ ID NO: 12 - protein, 6 aa, synthetic construct
  REGION 1..6: note = synthetic
QAIRND

SEQ ID NO: 13-14 - skipped (2 entries)

SEQ ID NO: 15 - DNA, 27 bp, synthetic construct
  misc_feature 1..27: note = synthetic
  source 1..27: mol_type = other DNA
caacagtataatagatacccgtggacg

SEQ ID NO: 16 - protein, 9 aa, synthetic construct
  REGION 1..9: note = synthetic
QQYNRYPWT

SEQ ID NO: 17 - DNA, 847 bp, synthetic construct
  misc_feature 1..847: note = synthetic
  source 1..847: mol_type = other DNA
agcgtccggacccaataacagttttaccaagggagcagctttctatcctggccacactgaggtgcatagcgtaatgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgagcattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtatgtgtgtgctcagcaggaaggctgtgagaagagcctgacctgccgacacgctccctccccctgccccttctacactctcctgggcc

SEQ ID NO: 18 - protein, 115 aa, synthetic construct
  REGION 1..115: note = synthetic
SSSHPIFHRGEFSVVSVWVGDKTTATDIKGKEVMVLGEVNINNSVFKQYFFETKCRDPNPVDSGCRGIDSKHWNSYCTTTHTFALTMDGKQAAWRFIRIDTACVCVLSRKAVRRA

(19) United States
(12) Patent Application Publication
(10) Pub. No.: US 2025/0011408 A1
(43) Pub. Date: Jan. 9, 2025

(54) METHODS AND COMPOSITIONS FOR REDUCING CENTRALIZED PAIN

(71) Applicant: Regeneron Pharmaceuticals, Inc., Tarrytown, NY (US)

(72) Inventors: Stephen DIMARTINO, Tarrytown, NY (US); Paula DAKIN, Tarrytown, NY (US); Gregory GEBA, Tarrytown, NY (US); Haitao GAO, Tarrytown, NY (US)

(21) Appl. No.: 18/710,939

(22) PCT Filed: Nov. 18, 2022
(86) PCT No.: PCT/US2022/080104

§ 371 Date: May 16, 2024

Related U.S. Application Data
(60) Provisional application No. 63/281,306, filed on Nov. 19, 2021.

(51) Int. Cl. C07K 16/22; A61K 39/00; A61P 19/02

(52) U.S. Cl. CPC C07K 16/22; A61P 19/02; A61K 2039/505; A61K 2039/545

(57) ABSTRACT

Methods and compositions for treating centralized pain are disclosed. Methods and compositions for reducing pain or improving physical function are disclosed. In certain aspects, the subject has centralized pain and osteoarthritis of the knee and/or hip and the anti-NGF antibody is fasinumab.

### RELATED APPLICATIONS

This application is a 35 U.S.C. § 371 filing of International Patent Application No. PCT/US2022/080104, filed Nov. 18, 2022, which claims the benefit of U.S. Provisional Patent Application Ser. No. 63/281,306, filed Nov. 19, 2021, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on May 14, 2024, is named 754651_RGN9-003US_ST26.xml and is 23,135 bytes in size.

### FIELD OF THE INVENTION

Disclosed herein are methods and compositions related to the treatment, reduction, or improvement of centralized pain or centralized pain disorders. Disclosed herein are methods and compositions related to the administration of nerve growth factor (NGF) antagonists, in particular a NGF antibody, to reduce centralized pain in a subject. Also disclosed herein are methods and compositions for use in reducing pain and or improving physical function in subjects with centralized pain.

### BACKGROUND

Centralized pain syndromes are associated with changes within the central nervous system that amplify peripheral input and/or generate the perception of pain in the absence of a noxious stimulus. Examples of idiopathic functional disorders that are often categorized as centralized pain syndromes include fibromyalgia, chronic pelvic pain syndromes, migraine, and temporomandibular disorder. Patients often suffer from widespread pain, associated with more than one specific syndrome, and report fatigue, mood and sleep disturbances, and poor quality of life. Clauw D J. (2015) Mayo Clin. Proc. 90:680-692. The high degree of symptom comorbidity and a lack of definitive underlying etiology make these syndromes notoriously difficult to treat.

Furthermore, patients with problems controlling centralized pain may also have difficulty controlling other types of pain, such as osteoarthritis (OA) joint pain and pre-op surgical pain for knee and hip arthroplasty. Brummet et al. (2015) Arthritis Rheum 67(5): 1386-1394. As such, patients with centralized pain may be a surrogate for overall poorer analgesic outcomes.

There remains an unmet medical need for alternative pain relief treatment options for subjects with centralized pain or centralized pain disorders, including those with fibromyalgia. Pharmacological treatments such as nonsteroidal anti-inflammatory drugs (NSAIDs) are widely used in the treatment of fibromyalgia and other centralized pain disorders, despite the lack of evidence of efficacy of pain relief for these conditions. Fasinumab is a fully-human high-affinity monoclonal antibody directed against NGF that has the potential to effectively reduce centralized pain in subjects.

### SUMMARY OF THE DISCLOSURE

In one aspect, disclosed herein are compositions and methods for treating, reducing, or improving centralized pain. In some embodiments, disclosed herein are methods for reducing centralized pain. In some embodiments, the methods comprise selecting a subject with centralized pain. In some embodiments, the methods comprise administering to a subject an anti-human nerve growth factor (NGF) antibody or an antigen-binding fragment thereof, or a pharmaceutical composition comprising the anti-human NGF antibody or antigen-binding fragment thereof. In some embodiments, the methods comprise administering an anti-human NGF antibody or antigen-binding fragment thereof that comprises a heavy chain complementarity determining region (HCDR)1 comprising or consisting of the amino acid sequence of SEQ ID NO: 4, an HCDR2 comprising or consisting of the amino acid sequence of SEQ ID NO:6, an HCDR3 comprising or consisting of the amino acid sequence of SEQ ID NO:8, a light chain complementarity determining region (LCDR)1 comprising or consisting of the amino acid sequence of SEQ ID NO:12, an LCDR2 comprising or consisting of the amino acid sequence AAF, and an LCDR3 comprising or consisting of the amino acid sequence of SEQ ID NO: 16. In some embodiments, the methods comprise administering to a subject a pharmaceutical composition comprising an anti-human NGF antibody comprising a heavy chain variable region (HCVR)/light chain variable region (LCVR) amino acid sequence pair of SEQ ID NOs: 2/10. In some embodiments, the subject is an adult. In some embodiments, the subject suffers from fibromyalgia. In some embodiments, the subject suffers from osteoarthritis (OA).

In one aspect of the disclosed methods for reducing centralized pain, the subject suffers from a high level of centralized pain. In some embodiments, the high level of centralized pain is measured or defined by a Survey of Pain Classification (QSPC) score. In some embodiments, the QSPC score is the score from the 2011 diagnostic criteria for fibromyalgia from the American College of Rheumatology (referred to herein as “FM score”), as described in Wolfe et al. (2011) J Rheumatol 38(6): 1113-22. In some embodiments, the subject has a FM score of 9 or above prior to treatment with the pharmaceutical composition comprising the anti-NGF antibody. In some embodiments, the subject has a FM score of 13 or above prior to treatment with the pharmaceutical composition comprising the anti-NGF antibody.

In one aspect of the disclosed methods, the efficacy of the NGF antibody is measured by the change from baseline of a centralized pain score for the subject. In some embodiments, the centralized pain score is a QSPC score. In some embodiments, the centralized pain score is the FM score for the subject.

In one aspect of the disclosed methods, the efficacy of the NGF antibody is measured by the change from baseline of a generalized pain score for the subject. In some embodiments, the generalized pain score is determined from a score from one or more questions from the Short Form Health Survey (SF-36). In some embodiments, the generalized pain score is a SF-36 item 7 (bodily pain) score. In some embodiments, the generalized pain score is a SF-36 item 8 (pain interference) score. In some embodiments, the generalized pain score is a EuroQoL 5 Dimensions 5 Level Questionnaire (EQ-5D-5L) score. In some embodiments, the generalized pain score is an EQ-5D-5L item 4 (pain/discomfort) score.

In one aspect, disclosed herein are compositions and methods for reducing pain or improving physical function. In some embodiments, the methods comprise selecting a subject suffering from osteoarthritis of the knee or hip and centralized pain. In some embodiments, the methods comprise administering to a subject a pharmaceutical composition comprising an anti-human nerve growth factor (NGF) antibody that binds specifically to NGF, wherein the anti-NGF antibody or the antigen binding fragment thereof comprises a heavy chain variable region (HCVR)/light chain variable region (LCVR) amino acid sequence pair of SEQ ID NOs: 2/10. In some embodiments, the subject is an adult. In some embodiments, the subject suffers from fibromyalgia.

In one aspect of the disclosed methods for reducing pain or improving physical function, the subject suffers from a high level of centralized pain. In some embodiments, the high level of centralized pain is measured or defined by a Survey of Pain Classification (QSPC) score. In some embodiments, the QSPC score is the score from the 2011 diagnostic criteria for fibromyalgia from the American College of Rheumatology (referred to herein as “FM score”), as described in Wolfe et al. (2011) J Rheumatol 38(6): 1113-22. In some embodiments, the subject has a FM score of 9 or above prior to treatment with the pharmaceutical composition comprising the anti-NGF antibody. In some embodiments, the subject has a FM score of 13 or above prior to treatment with the pharmaceutical composition comprising the anti-NGF antibody.

In one aspect of the disclosed methods, the efficacy of the NGF antibody in reducing pain or improving physical function is measured by the change from baseline of a pain-associated parameter. In some embodiments, the pain-associated parameter is the Western Ontario and McMaster Universities Osteoarthritis Index (WOMAC) pain subscale score; the WOMAC physical function subscale score; or the Patient Global Assessment (PGA) score. In some embodiments, the pain-associated parameter is the Numeric Rating Scale of the average walking index joint pain, the EuroQoL 5 Dimensions 5 Level Questionnaire, the 36-item Short Form Survey, the Healthcare Resource Utilization Questionnaire, the Work Productivity and Activity Impairment-Osteoarthritis, or the Treatment Satisfaction Questionnaire for Medication.

In some embodiments of the disclosed methods, the NGF antibody is administered at a dose of about 1 mg every 4 weeks (Q4W). In some embodiments of the disclosed methods, the NGF antibody is administered at a dose of about 3 mg every 4 weeks (Q4W). In some embodiments of the disclosed methods, the NGF antibody is administered at a dose of about 6 mg every 4 weeks (Q4W). In some embodiments of the disclosed methods, the NGF antibody is administered at a dose of about 9 mg every 4 weeks (Q4W).

In one aspect, disclosed herein is a pharmaceutical composition comprising an anti-NGF antibody comprising three heavy chain complementarity determining region (HCDR) sequences (HCDR1, HCDR2, HCDR3) comprising SEQ ID NOs: 4, 6 and 8, respectively, and three light chain complementarity determining (LCDR) sequences (LCDR1, LCDR2, LCDR3) comprising SEQ ID NOS: 12, 14 and 16, respectively. The pharmaceutical composition can be used according to any of the above aspects and/or embodiments.

In one aspect, disclosed herein is a pharmaceutical composition comprising a NGF antibody, wherein the NGF antibody is fasinumab. The pharmaceutical composition can be used according to any of the above aspects and/or embodiments.

In another aspect, disclosed herein are anti-NGF antibodies or antigen-binding fragments thereof for use in reducing centralized pain, e.g., in a subject having a centralized pain disorder or in a subject having a centralized pain score that is above a threshold value (e.g., a score of ≥9 on the 2011 ACR fibromyalgia diagnostic criteria score (“FM score”)). In some embodiments, the anti-NGF antibody or antigen-binding fragment thereof comprises an HCDR1comprising or consisting of the amino acid sequence of SEQ ID NO:4, an HCDR2 comprising or consisting of the amino acid sequence of SEQ ID NO:6, an HCDR3 comprising or consisting of the amino acid sequence of SEQ ID NO:8, an LCDR1 comprising or consisting of the amino acid sequence of SEQ ID NO:12, an LCDR2 comprising or consisting of the amino acid sequence AAF, and an LCDR3 comprising or consisting of the amino acid sequence of SEQ ID NO: 16.

In another aspect, disclosed herein are anti-NGF antibodies or antigen-binding fragments thereof for use in the preparation of a medicament for reducing centralized pain, e.g., in a subject having a centralized pain disorder or in a subject having a centralized pain score that is above a threshold value (e.g., a score of ≥9 on the 2011 ACR fibromyalgia diagnostic criteria score (“FM score”)). In some embodiments, the anti-NGF antibody or antigen-binding fragment thereof comprises an HCDR1 comprising or consisting of the amino acid sequence of SEQ ID NO: 4, an HCDR2 comprising or consisting of the amino acid sequence of SEQ ID NO:6, an HCDR3 comprising or consisting of the amino acid sequence of SEQ ID NO:8, an LCDR1 comprising or consisting of the amino acid sequence of SEQ ID NO:12, an LCDR2 comprising or consisting of the amino acid sequence AAF, and an LCDR3 comprising or consisting of the amino acid sequence of SEQ ID NO: 16.

Each of the above aspects are also disclosed and described herein as corresponding uses and methods of treatment.

### BRIEF DESCRIPTION OF THE FIGURES

FIG. 1: The 2011 ACR fibromyalgia diagnostic criteria/survey of pain classification (QSPC) as described in Wolfe et al. (2011) J Rheumatol 38(6): 1113-22.

FIG. 2: Change from baseline in WOMAC pain subscale score for subjects with FM scores/QSPC scores of 0 to 4. For all subjects, reported FM scores/QSPC scores were collected on day 1 of the treatment period.

FIG. 3: Change from baseline in WOMAC pain subscale score for subjects with FM/QSPC scores of 5 to 8.

FIG. 4: Change from baseline in WOMAC pain subscale score for subjects with FM/QSPC scores of 9 and above (i.e. 9 to 31).

FIG. 5: Change from baseline in the 36-item Short Form Survey (SF-36) item 7 score for subjects with FM/QSPC scores of 0 to 4.

FIG. 6: Change from baseline in the SF-36 item 7 score for subjects with FM/QSPC scores of 5 to 8.

FIG. 7: Change from baseline in the SF-36 item 7 score for subjects with FM/QSPC scores of 9 and above (i.e. 9 to 31).

FIG. 8: Change from baseline in the SF-36 item 8 score for subjects with FM/QSPC scores of 0 to 4.

FIG. 9: Change from baseline in the SF-36 item 8 score for subjects with FM/QSPC scores of 5 to 8.

FIG. 10: Change from baseline in the SF-36 item 8 score for subjects with FM/QSPC scores of 9 and above (i.e. 9 to 31).

FIG. 11: Change from baseline in the EQ-5D-5L item 4 score for subjects with FM/QSPC scores of 0 to 4.

FIG. 12: Change from baseline in the EQ-5D-5L item 4 score for subjects with FM/QSPC scores of 5 to 8.

FIG. 13: Change from baseline in the EQ-5D-5L item 4 score for subjects with FM/QSPC scores of 9 and above (i.e. 9 to 31).

### DETAILED DESCRIPTION

Before the invention disclosed herein is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

All patents, applications and non-patent publications mentioned herein are incorporated herein by reference in their entireties.

### Centralized Pain Disorders

The construct of “centralized pain” refers to central nervous system mechanisms associated with augmented pain and sensory processing. Clinically, it manifests as widespread chronic pain often independent of nociceptive input, combined with fatigue, sleep, cognitive, and mood problems. (Neville et al. (2018) Clin J Pain. 34(10): 909-917). Centralized pain disorders are often comorbid with one another and are accompanied by fatigue, mood and sleep disturbances, and poor quality of life scores (Clauw D J. (2015) Mayo Clin. Proc. 90:680-692).

Examples of centralized pain disorders include fibromyalgia, chronic pelvic pain syndromes (irritable bowel syndrome (IBS), interstitial cystitis/painful bladder syndrome (IC/PBS), vulvodynia, and chronic prostatitis/chronic pelvic pain syndrome (CP/CPPS)), migraine, chronic fatigue syndrome (CFS), and temporomandibular disorder (Clauw, 2015). These disorders have a high degree of co-occurrence and are generally accompanied by fatigue, sleep problems, and cognitive difficulties. Other chronic pain states are often associated with centralized pain disorders, including osteoarthritis, rheumatoid arthritis, lupus, and irritable bowel syndrome. Chronic pain conditions with centralized phenomena are not well understood.

Fibromyalgia (FM) is the prototypical centralized pain disorder. Patients with fibromyalgia have chronic (>3 months) pain that is generalized; occurs in multiple sites; and is associated with fatigue, sleep problems, and cognitive or somatic symptoms. Bair et al. (2020) Ann Internal Med. 172(5):ITC33-48. The source of pain of FM remains unclear but may result from neurochemical imbalances in the central nervous system (CNS) that lead to an augmentation of pain perception, typified by allodynia (pain due to a stimulus that does not usually provoke pain) and hyperalgesia (increased pain from a stimulus that usually provokes pain). Similar findings of hyperalgesia and allodynia have been observed in other chronic pain states, including irritable bowel syndrome, female urethral syndrome or overactive bladder, temporomandibular joint syndrome, myofascial pain syndrome, and even osteoarthritis (OA). (Arnold et al. (2016) Clin J Pain, Vol. 32:737-46).

Diagnosis of centralized pain disorders remains difficult because it is entirely dependent on patient report of symptoms and functional impairment, without any defining physical or laboratory abnormality. Although the diagnosis is typically based on clinical evaluation, various criteria have been developed, tested in population based studies, and disseminated to aid in clinical diagnosis. Despite the availability of these criteria, no international consensus guidelines on diagnosis and management of centralized pain disorders currently exist.

There is currently no “gold standard” for treatment of centralized pain disorders. The overall approach to treating centralized pain disorders is focused on maintaining or improving physical function, improving quality of life, and managing symptoms. Pharmacological treatments may be helpful for some patients with centralized pain, however, overall patient response has been highly inconsistent and moderate at best. Although the traditional pharmacological treatment paradigm begins with the use of simple analgesics and tricyclic antidepressants, other agents that may be used include other antidepressants, gabapentinoids, dopaminergic agents, opiods, and sleep modifiers. NSAIDs are widely used in the treatment of fibromyalgia, despite the lack of evidence that NSAIDs are beneficial for pain reduction or any other outcome. (Derry S et al. (2017) Cochrane Database of Systematic Rev. Issue 3, Art. No. CD012332. Indeed, centralized pain disorders, including fibromyalgia, present a clinical challenge because the cause of, the ideal treatment, and any potential cures are unknown. (Fitzcharles, M A et al. (2013) Pain Res Management Vol 18(3): 119-126).

### Methods for Reducing Pain or Improving Physical Function

In one aspect, disclosed herein are methods for reducing pain or improving physical function in a subject. In some embodiments, the reduction in pain or improvement in physical function is measured by one or more pain-associated parameters. Further disclosed herein are methods for improving one or more pain-associated parameters in a subject in need thereof, wherein the methods comprise administering an NGF antagonist or a pharmaceutical composition comprising an NGF antagonist, e.g., an anti-NGF antibody of the disclosure or an antigen binding fragment thereof, to the subject.

Examples of “pain-associated parameters” include: (a) Western Ontario and McMaster Universities Osteoarthritis Index (WOMAC) pain score; (b) WOMAC physical function subscale scores; (c) Patient Global Assessment (PGA) score; (d) the knee and/or hip pain Numerical Rating Scale (NRS) score; (e) the Roland Morris Disability Questionnaire (RMDQ) total score; (f) the Medical Outcomes Study (MOP) sleep subscale score; (g) the short form health survey (SF-36) subscale scores; and (h) the EuroQoL 5 Dimensions 5 Level Questionnaire (EQ-5D-5L). Pain-associated parameters also include centralized pain scores.

As used herein, the term “baseline,” with regard to a pain-associated parameter, means the value of the centralized pain score or other pain-associated parameter for a subject or group of subjects prior to or at the time of administration of the NGF antagonist, e.g., a pharmaceutical composition comprising an anti-NGF antibody or fragment thereof.

To determine whether a pain-associated parameter has “improved,” the parameter is quantified at baseline and at one or more time points after administration of the pharmaceutical composition disclosed herein. For example, a pain-associated parameter may be measured at various time points after administration of the anti-NGF antibody, e.g., at day 1, day 2, day 3, day 4, day 5, day 6, day 7, day 8, day 9, day 12, day 18, day 22, day 36, day 50, day 57, day 64, day 78, day 85, day 92, day 106, day 113, day 120; or at the end of week 1, week 2, week 3, week 4, week 5, week 6, week 7, week 8, week 9, week 10, week 11, week 12, week 13, week 14, week 15, week 16, week 24, week 36, week 44, week 52, week 72, week 104, or longer, after the initial treatment with the anti-NGF antibody. The difference between the value of the parameter at a particular time point following initiation of treatment and the value of the parameter at baseline is used to establish whether there has been an “improvement” (e.g., a decrease) in the pain associated parameter. For example, disclosed herein are therapeutic methods which result in an improvement from baseline of at least about 10%, 20%, 30%, 40%, 50%, or more in a pain-associated parameter following administration of about 1 mg of fasinumab).

In one aspect, following administration of a pharmaceutical composition comprising fasinumab, the subject exhibits an improvement in one or more pain-associated parameters. In some embodiments, the subject exhibits an improvement in one or more of: (a) Western Ontario and McMaster Universities Osteoarthritis Index (WOMAC) pain score; (b) WOMAC physical function subscale scores; and (c) Patient Global Assessment (PGA) score. In some embodiments, the efficacy of the disclosed treatments in reducing pain and/or improving physical function is measured by the change from baseline of: (1) the Numeric Rating Scale (NRS) of the average walking index for joint pain, (2) the EuroQoL 5 Dimensions 5 Level Questionnaire, (3) the 36-item Short Form Survey (SF-36), (4) the Healthcare Resource Utilization Questionnaire, (5) the Work Productivity and Activity Impairment Osteoarthritis, or (6) the Treatment Satisfaction Questionnaire for Medication.

In one aspect, the methods disclosed herein result in reducing pain in a subject. In some embodiments, the methods disclosed herein result in an improvement in a pain-associated parameter. In some emodiments, the pain-associated parameter is the WOMAC pain subscale score. The WOMAC pain subscale score is a composite index of 5 questions related to joint pain while walking, using stairs, at rest in bed, sitting or lying, and standing and is described in Bellamy N. WOMAC Osteoarthritis Index: A User's Guide. London, Ontario, Canada: Victoria Hospital; 1995. Individual WOMAC questions are scored on a scale of 0-10. The scores from each of the 5 questions are averaged. In certain aspects, the improvement demonstrated by the treatment disclosed herein includes an improvement from baseline in the WOMAC pain subscale score.

In one aspect, the methods disclosed herein result in improving physical function in a subject. In some embodiments, the methods disclosed herein result in an improvement in the WOMAC physical function subscale score. The WOMAC physical function subscale score measures 17 items for functional limitation (scale of 0-10). Physical functioning questions cover everyday activities such as stair use, standing up from a sitting or lying position, standing, bending, walking, getting in and out of a car, shopping, putting on or taking off socks, lying in bed, getting in or out of a bath, sitting, and heavy and light household duties. In certain aspects, the improvement demonstrated by the treatment disclosed herein includes an improvement from baseline in the WOMAC physical function subscale score.

### Methods for Reducing Centralized Pain

In one aspect, disclosed herein are methods for reducing centralized pain in a subject. In some embodiments, the reduction in centralized pain is measured by one or more centralized pain scores. In some embodiments, the methods disclosed herein result in an improvement in one or more centralized pain scores in a subject. In some embodiments, centralized pain in a subject is measured using a score from a Survey of Pain Classification (QSPC).

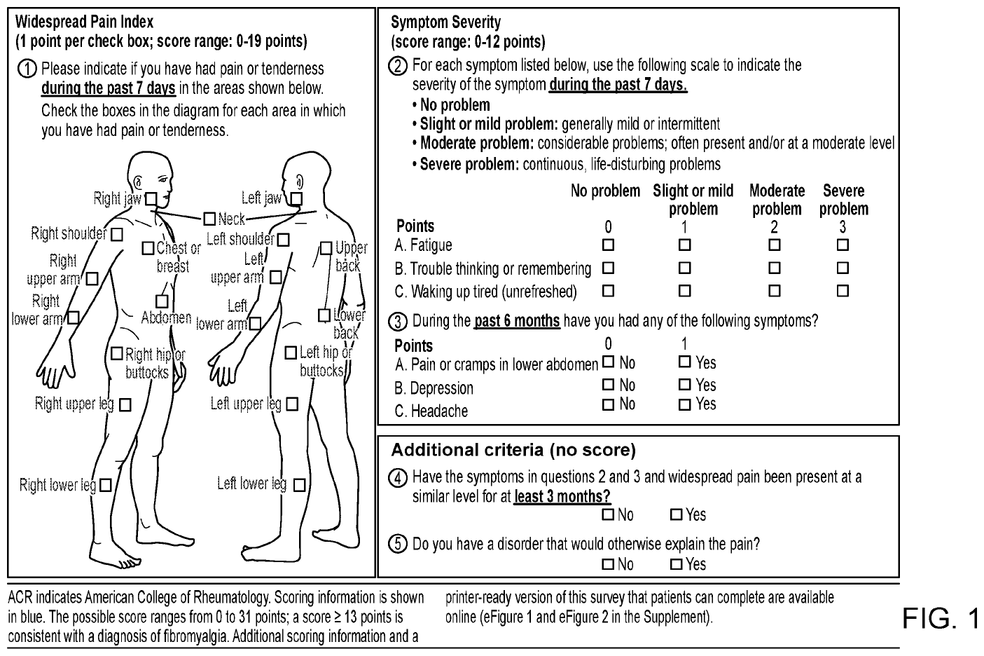

In some embodiments, the QSPC is the 2011 diagnostic criteria from the American College of Rheumatology (ACR) described in Wolfe et al. (2011) J Rheumatol 38(6): 1113-22. This QSPC is comprised of the Widespread Pain Index (WPI) and the Symptom Severity score (SS score). The WPI assesses 19 body locations to determine the spatial distribution of pain in the past week, with scores ranging 0-19. The SS score evaluates the severity of three cardinal FM symptoms (fatigue, trouble thinking or remembering, and waking up tired/unrefreshed), as well as the presence of three additional somatic symptoms (pain or cramps in lower abdomen, depression, and headache). The SS score ranges 0-12. The WPI and SS score are combined to calculate an overall index of FM severity score ranging 0-31 (referred to herein as “FM score”). A FM score of ≥13 is associated with fibromyalgia positive classification. The 2011 ACR diagnostic criteria (QSPC) is depicted in FIG. 1. In some embodiments, the methods disclosed herein result in an improvement in a QSPC score for a subject. In some embodiments, the methods disclosed herein result in an improvement in the FM score of a subject.

It has been hypothesized that the 2011 ACR fibromyalgia QSPC score may be useful for identifying OA patients with centralized pain. (Neville et al. (2018) Clin J Pain. 34(10): 909-917). A significant disparity exists between reported pain in OA and identifiable joint pathology on radiographic imaging: some patients reporting pain have minimal findings on imaging, whereas others have extensive joint pathology on imaging but report little pain, suggesting that at least a subset of knee OA patients have predominantly centralized pain. The 2011 ACR QSPC score has also been reported to assist with predicting long-term analgesic outcomes following total knee and hip arthroplasty. Brummett et al. (2015) Arthritis Rheum 67(5): 1386-1394.

As used herein, the term “generalized pain score” refers to any score or value that represents pain not limited to any specific part of the body. In some embodiments, a generalized pain score refers to the overall level or cumulativel level of pain in a patient. As disclosed herein, a generalized pain score may be used as a predictor or proxy for a centralized pain score (e.g. a FM score) in a subject. Non-limiting examples of generalized pain scores are scores from one or more questions or items from the 36-item short form health survey (SF-36), the EuroQoL 5 Dimensions 5 Level Questionnaire (EQ-5D-5L), or any other survey of general health. In some embodiments, the generalized pain score is a SF-36 item 7 (bodily pain) score. In some embodiments, the generalized pain score is a SF-36 item 8 (pain interference) score. In some embodiments, the generalized pain score is an EQ-5D-5L item 4 (pain/discomfort) score.

In some embodiments, a generalized pain score in a subject is determined using a score from the 36-item short form health survey (SF-36). Briefly, the SF-36 is a self-administered survey of general health (Ware et a. (1992) Med Care; 30:473-83). It measures 8 domains of health: physical functioning, role limitations due to physical health, bodily pain, general health perceptions, vitality, social functioning, role limitations due to emotional problems, and mental health. It yields scale scores for each of these 8 health domains, and 2 summary measures of physical and mental health: the physical component summary and the mental component summary. Each scale is directly transformed into a 0-100 scale on the assumption that each question carries equal weight. The lower the score the more disability. The higher the score the less disability i.e., a score of zero is equivalent to maximum disability and a score of 100 is equivalent to no disability.

In some embodiments, the methods disclosed herein result in an improvement in a generalized pain score in a subject. In some embodiments, the methods disclosed herein result in an improvement in a SF-36 score in a subject. In some embodiments, the methods disclosed herein result in an improvement of the SF-36 item 7 (bodily pain) score for a subject. In some embodiments, the methods disclosed herein result in an improvement of the SF-36 item 8 (pain interference) score for a subject.

In some embodiments, a generalized pain score in a subject is determined using a score from the EuroQOL 5 Dimensions 5 Level Questionnaire (EQ-5D-5L). The EQ-5D-5L is a standardized measure of health status developed by the EuroQoL Group to provide a simple, generic measure of health for clinical and economic appraisal (van Reenen M, Janssen B. EQ-5D-5L User Guide. Version 2.1. Rotterdam, The Netherlands: European Quality of Life Research Foundation; April 2015). The EQ-5D-5L, as a measure of health-related quality of life, defines health in terms of 5 dimensions: mobility, self-care, usual activities, pain/discomfort, anxiety/depression. Each dimension has 3 ordinal levels of severity: “no problem” (1), “some problems” (2), “severe problems” (3). Overall health state is defined as a 5-digit number. Health states defined by the 5-dimensional classification can be converted into corresponding index scores that quantify health status, where −0.594 represents “severe problems” and 1 represents “no problem.”

In some embodiments, the methods disclosed herein result in an improvement in a EQ-5D-5L score in a subject. In some embodiments, the methods disclosed herein result in an improvement of item 4 (pain/discomfort) of the EQ-5D-5L.

Centralized pain in a subject may be measured using any other methods that are used to measure pain intensity. Various instruments have been developed to evaluate pain intensity (how much a person hurts) and pain affect (how much a person suffers). Three methods have traditionally been used to measure pain intensity: visual analogue scales (VASs), verbal rating scales (VRSs), and numerical rating scales (NRSs). See Von Korff M et al. (2000), Spine 25:3140-3151; Zanoli G et al. (2000), Spine 25:3178-3185; Haefeli M and Elfering A (2006), Eur Spine J 15 (Suppl 1): S17-S24; McGuire DB (1999), Instruments for Health-Care Research 528-561 (Eds Frank-Stromborg M and Olsen S) Boston: Jones and Bartlett; Ogon M et al. (1996), Pain 64:425-428; Hägg O et al. (2003), Eur Spine J 12:12-20; Jensen M P et al. (1986), Pain 27:117-126).

The visual analogue scale (VAS) consists of a line, usually 100 mm long, whose ends are labeled as the extremes (‘no pain’ and ‘pain as bad as it could be’); the rest of the line is blank. The patient is asked to put a mark on the line indicating their pain intensity (at the present time, over the past week, or over the past 2 weeks, etc.). The distance between that mark and the origin is measured to obtain the patient's score. Sometimes descriptive terms, such as ‘mild’, ‘moderate’ and ‘severe’, or numbers are provided along the scale for guidance, with “moderate” falling within the mid-range of the scale and the scale is then referred to as a graphic rating scale.

Verbal rating scales (VRSs) consist of a list of adjectives that describe different levels of pain intensity. A VRS for pain includes adjectives that reflect the extremes (e.g. ‘no pain’ to ‘pain as bad as it could be’), and sufficient adjectives to capture the gradations in between. VRSs are most frequently five-point or six-point scales. The patient is asked to select in a questionnaire or state verbally the adjective that best describes his or her level of pain intensity. In behavioral rating scales, different pain levels are described by sentences including behavioral parameters.

Additional details for various methods for assessing pain, non-responsiveness, or intolerance to analgesics and the like are disclosed in US20180147280, which is herein incorporated by reference in its entirety.

### Methods of Treating Subjects with Centralized Pain

Disclosed herein are methods and compositions for treating patients with centralized pain. These patients represent a patient population with an unmet medical need for pain relief therapy. These patients may benefit from treatment with an NGF antagonist such as fasinumab, which has the potential to be both safe and effective in this difficult to treat patient population.

As used herein, the terms “treat”, “treating”, or the like, mean to alleviate symptoms, eliminate the causation of symptoms either on a temporary or permanent basis, or to prevent or slow the appearance of symptoms of the named disorder or condition. In certain embodiments, the symptom of the disorder or condition is pain.

In one aspect, disclosed herein are methods and compositions for the management of high levels of centralized pain in a subject. In some embodiments, the centralized pain is associated with or accompanied by OA pain.

Disclosed herein are methods comprising administering to a subject a therapeutic composition comprising an NGF antagonist. In some embodiments, a “subject” refers to a human that exhibits centralized pain. In some embodiments, a “subject” refers to a patient diagnosed with fibromyalgia. In some embodiments, a “subject” refers to a patient suffering from centralized pain. In some embodiments, the subject has a history of inadequate pain relief from standard analgesic therapy (e.g., no significant pain reduction after administration of the standard analgesic therapy for an average of 4 days/week during a 4 week period), or intolerance to standard analgesic therapy.

The term “subject” may also include, e.g., subjects who, prior to treatment, exhibit (or have exhibited) one or more pain-associated parameters which are improved following treatment with an anti-NGF antibody. Non-limiting examples of pain-associated parameters include a centralized pain score and a generalized pain score. In some embodiments, the anti-NGF antibody is fasinumab.

In one aspect, the methods disclosed herein include administering a pharmaceutical composition at a specific dose and frequency. In some embodiments, the anti-NGF antibody or antigen binding fragment thereof is administered in an amount from 1 mg to 10 mg, administered every week, every two weeks, every three weeks, every four weeks, every five weeks, every six weeks, every seven weeks, or every eight weeks. In some embodiments, the specific dose is 1 mg of the anti-NGF antibody or an antigen binding fragment thereof. As shown by the clinical studies described herein, this dose of about 1 mg of the anti-NGF antibody or an antigen binding fragment thereof provides an improvement in pain (i.e. a reduction in pain) and/or an improvement in physical function in a subject suffering from OA of the knee or hip. In some embodiments, the subject suffers from pain defined as moderate to severe. In some embodiments, the subject suffers from centralized pain. In some embodiments, the subject suffers from centralized pain defined as high level centralized pain. In some embodiments, the subject is intolerant to one or more analgesic treatments and/or the subject (i.e. the subject's pain) is non-responsive to one or more analgesic treatments.

In some embodiments, the NGF antibody is administered at a dose of about 1 mg every 4 weeks (Q4W). In some embodiments, the NGF antibody is administered at a dose of about 3 mg every 4 weeks (Q4W). In some embodiments, the NGF antibody is administered at a dose of about 6 mg every 4 weeks (Q4W). In some embodiments, the NGF antibody is administered at a dose of about 9 mg every 4 weeks (Q4W). In some embodiments, the NGF antibody is administered at a dose of about 1 mg every 8 weeks (Q8W).

As noted above, disclosed herein are methods to treat centralized pain in subjects who exhibit a history of inadequate pain relief, or intolerance to, standard analgesic therapy, or who are resistant, non-responsive, or inadequately responsive to treatment with a standard analgesic. The term “inadequate pain relief” refers to an unacceptable level of pain relief experienced by subjects after pain relief treatment, such as treatment with a standard analgesic. For example, subjects with inadequate pain relief may find that they cannot go about conducting normal daily activities due to the pain level index. The term “inadequate pain relief” also refers to an unacceptable reduction in pain and/or unacceptable improvement in pain after pain relief treatment, such as treatment with a standard analgesic.

The term “intolerance to standard analgesic therapy” (or treatments) refers to subjects or patients who exhibit an adverse event or side effect after treatment with the standard analgesic, such as for example an allergic reaction to a standard analgesic. The term “resistant, non-responsive, or inadequately responsive to a standard analgesic”, as used herein, refers to subjects or patients with knee and/or hip pain who have been treated with for example, an NSAID, and wherein the NSAID does not have a therapeutic effect. In some embodiments, the term refers to reduced patient compliance and/or toxicity and side effects and/or ineffectiveness of the administered analgesic to reduce, ameliorate or decrease the symptoms of knee and/or hip pain. In some embodiments, the patients who are “resistant, non-responsive or inadequately responsive to a standard analgesic” may show no improvement in one or more pain-associated parameters. Examples of pain-associated parameters are described elsewhere herein.

### NGF Antagonists

As disclosed in detail above, disclosed herein are methods comprising administering to a subject in need thereof a therapeutic composition comprising an NGF antagonist. As used herein, an “NGF antagonist” is any agent, which binds to or interacts with NGF and inhibits the normal biological function of NGF when NGF is expressed on a cell in vitro or in vivo. NGF antagonists disclosed herein encompass any agent that blocks the interaction of NGF with one or both of its receptors, TrkA and p75. Non-limiting examples of categories of NGF antagonists include small molecule NGF antagonists, anti-NGF aptamers, peptide-based NGF antagonists (e.g., “peptibody” molecules), and mRNA-based NGF antagonists. NGF antagonists disclosed herein also encompass antibodies or antigen-binding fragments of antibodies that specifically bind human NGF (referred to herein as an “anti-NGF antibody” or “NGF antibody). In some embodiments, the NGF antagonist is fasinumab. In some embodiments, the NGF antagonist is tanezumab.

Administration of NGF has been shown to provoke pain in both rodents (Lewin, G. R., et.al., (1994), Eur. J. Neurosci 6:1903-1912) and humans (McArthur, J. C., et.al., (2000), Neurology 54:1080-1088), while NGF antagonists have been shown to prevent hyperalgesia and allodynia in animal models of neuropathic and chronic inflammatory pain (Ramer, M. S. et.al., (1999) Eur J Neurosci 11:837-846). Humans with mutations in TrkA (hereditary sensory and autonomic neuropathy IV) or NGF (hereditary sensory and autonomic neuropathy V) have been identified with a loss of deep pain perception (Indo, Y. et.al., (1996), Nature Genetics, 13:485-488), Einarsdottir, E., et.al., (2004), Human Molecular Genetics 13:799-805). In addition, NGF is known to be elevated in the synovial fluid of patients with rheumatoid arthritis and other types of arthritis (Aloe, L. et.al., (1992), Arthritis Rheum 35:351-355; Halliday, D. A., (1998), Neurochem Res. 23:919-922), and to be up-regulated in injured and inflamed tissues in conditions such as cystitis, prostatitis, and chronic headache (Lowe, E. M., et.al., (1997), Br. J. Urol. 79:572-577; Miller, L. J., et.al., (2002), Urology 59:603-608; Sarchielli, P. et. al., (2001), Neurology 57:132-134).

The terms “NGF,” “hNGF,” and the like, as used herein, are intended to refer to nerve growth factor, and in particular, to human nerve growth factor, the amino acid sequence of which is shown as SEQ ID NO: 18 and which is encoded by the nucleic acid sequence shown as SEQ ID NO: 17 (see Table 15, below). Unless specifically designated as being from a non-human species, the term “NGF”, as used herein, shall be understood to mean human NGF.

The term “antibody,” as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-NGF antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; (Vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20,40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody of the present disclosure using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “human antibody”, as used herein, is intended to include non-naturally occurring human antibodies. The term includes antibodies that are recombinantly produced in a non-human mammal, or in cells of a non-human mammal. The term is not intended to include antibodies isolated from or generated in a human subject.

The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, C_{H}2 or C_{H}3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” NGF, as used in the context of the present disclosure, includes antibodies that bind NGF or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, less than about 0.5 nM, less than 0.1 nM, less than 1.0 pM, or less than 0.5 pM, as measured in a surface plasmon resonance assay. An isolated antibody that specifically binds human NGF may, however, have cross-reactivity to other antigens, such as NGF molecules from other (non-human) species.

The anti-NGF antibodies useful for the methods of the present disclosure may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present disclosure includes methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present disclosure.

The present disclosure also includes methods involving the use of anti-NGF antibodies comprising variants of any of the HCVR, LCVR, and/or CDR amino acid sequences disclosed herein having one or more conservative substitutions. For example, the present disclosure includes the use of anti-NGF antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance,” as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “KD,” as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

### Preparation of Human Antibodies

Methods for generating human antibodies in transgenic mice are known in the art. Any such known methods can be used in the context of the present disclosure to make human antibodies that specifically bind to human NGF.

Using VELOCIMMUNE™ technology (see, for example, U.S. Pat. No. 6,596,541, Regeneron Pharmaceuticals) or any other known method for generating monoclonal antibodies, high affinity chimeric antibodies to NGF are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc, using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

Specific examples of human antibodies or antigen-binding fragments of antibodies that specifically bind NGF which can be used in the context of the methods of the present disclosure include any antibody or antigen-binding fragment which comprises the three heavy chain CDRs (HCDR1, HCDR2 and HCDR3) contained within a heavy chain variable region (HCVR) having an amino acid sequence consisting of SEQ ID NO: 2. The antibody or antigen-binding fragment may comprise the three light chain CDRs (LCVR1, LCVR2, LCVR3) contained within a light chain variable region (LCVR) having an amino acid sequence consisting of SEQ ID NO: 10. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments of the present disclosure, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 2/10.

In certain embodiments, the antibody or antigen-binding fragment thereof comprises an HCDR1 comprising or consisting of the amino acid sequence of SEQ ID NO:4, an HCDR2 comprising or consisting of the amino acid sequence of SEQ ID NO:6, an HCDR3 comprising or consisting of the amino acid sequence of SEQ ID NO:8, an LCDR1 comprising or consisting of the amino acid sequence of SEQ ID NO:12, an LCDR2 comprising or consisting of the amino acid sequence AAF, and an LCDR3 comprising or consisting of the amino acid sequence of SEQ ID NO: 16. In certain embodiments of the present disclosure, the antibody or antigen-binding fragment thereof comprises six CDRs (HCDR1/HCDR2/HCDR3/LCDR1/LCDR2/LCDR3) having the amino acid sequences consisting of SEQ ID NOs: 4/6/8/12/14/16.

In certain embodiments of the present disclosure, the antibody or antigen-binding fragment thereof comprises HCVR/LCVR amino acid sequence pairs consisting of SEQ ID NOS: 2/10.

As used herein, the term “Fasinumab” is used interchangeably to refer to an anti-NGF antibody. Fasinumab is a fully-human high-affinity monoclonal antibody directed against NGF (see U.S. Pat. No. 7,988,967 and PCT Publication No. WO 2009/023540 and WHO Drug Information Vol. 26, No. 2, (2012), which are all hereby incorporated by reference in their entirety). The amino acid sequences of the heavy chain and light chain variable regions and the CDRs portions as well as the nucleotide sequences of fasinumab are described in Tables 1A and 1B, respectively. The characterization of Fasinumab is described in PCT Publication No. WO 2009/023540 and WHO Drug Information Vol. 26, No. 2, (2012), which are all hereby incorporated by reference in their entirety.

TABLE 1A

| Antibody | AMINO ACID SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| Fasinumab | 2 | 4 | 6 | 8 | 10 | 12 | 14 | 16 |

TABLE 1B

| Antibody | NUCLEIC ACID SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| Fasinumab | 1 | 3 | 5 | 7 | 9 | 11 | 13 | 15 |

### Pharmaceutical Compositions

In some embodiments, the present disclosure includes methods, which comprise administering an NGF antagonist to a patient, wherein the NGF antagonist is contained within a pharmaceutical composition. The pharmaceutical compositions of the disclosure are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody administered to a patient according to the methods of the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-NGF antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351). Specific exemplary doses of anti-IL4R antibodies, and administration regimens involving the same, that can be used in the context of the present disclosure are disclosed elsewhere herein.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, Il and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-NGF antibody that can be used in the context of the present disclosure are disclosed. Examples of useful antibodies are disclosed in International Publication No. WO 2018/102294, U.S. Pat. No. 7,988,967, and U.S. Patent Application Publication No. 2012/0097565. Further, useful formulations comprising the antibodies are disclosed in U.S. Patent Application Publication No. US 2012/0014968. All of which are incorporated herein by reference.

### Dosage

The amount of NGF antagonist (e.g., anti-NGF antibody) administered to a subject according to the methods of the present disclosure is, generally, a therapeutically effective amount. As used herein, the phrase “therapeutically effective amount” means an amount of NGF antagonist that results in one or more of: (a) an improvement in one or more pain-associated parameters (as defined elsewhere herein); and/or (b) a detectable improvement in one or more symptoms or indicia of pain. A “therapeutically effective amount” also includes an amount of NGF antagonist that inhibits, prevents, lessens, or delays the progression of pain in a subject.

In the case of an anti-NGF antibody, a therapeutically effective amount can be from about 0.05 mg to about 600 mg, e.g., about 0.05 mg, about 0.1 mg, about 1.0 mg, about 1.5 mg, about 2.0 mg, about 3.0 mg, about 6.0 mg, about 9.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-NGF antibody. In some embodiments, a therapeutically effective amount can be from about 1 mg to about 10 mg of an anti-NGF antibody. In some embodiments, about 1 mg, about 3 mg, about 6 mg, or about 9 mg of an anti-NGF antibody is administered to a subject. In some embodiments, about 1 mg of an anti-NGF antibody is administered to a subject. In some embodiments, the anti-NGF antibody comprises three heavy chain complementarity determining region (HCDR) sequences (HCDR1, HCDR2, HCDR3) comprising SEQ ID NOs: 4, 6 and 8, respectively, and three light chain complementarity determining (LCDR) sequences (LCDR1, LCDR2, LCDR3) comprising SEQ ID NOs: 12, 14 and 16, respectively. In some embodiments, the anti-NGF antibody is fasinumab.

In some embodiments, the anti-NGF antibody comprising a heavy chain variable region (HCVR)/light chain variable region (LCVR) amino acid sequence pair of SEQ ID NOs: 2/10 is administered at a dose of about 1.0 mg. In some embodiments, the anti-NGF antibody comprising a heavy chain variable region (HCVR)/light chain variable region (LCVR) amino acid sequence pair of SEQ ID NOs: 2/10 is administered at a dose of about 1.0 mg about every 4 weeks (Q4W). In some embodiments, the anti-NGF antibody comprising a heavy chain variable region (HCVR)/light chain variable region (LCVR) amino acid sequence pair of SEQ ID NOs: 2/10 is administered at a dose of about 3.0 mg about every 4 weeks (Q4W). In some embodiments, the anti-NGF antibody comprising a heavy chain variable region (HCVR)/light chain variable region (LCVR) amino acid sequence pair of SEQ ID NOs: 2/10 is administered at a dose of about 6.0 mg about every 4 weeks (Q4W). In some embodiments, the anti-NGF antibody comprising a heavy chain variable region (HCVR)/light chain variable region (LCVR) amino acid sequence pair of SEQ ID NOs: 2/10 is administered at a dose of about 9.0 mg about every 4 weeks (Q4W). In some embodiments, the anti-NGF antibody is administered at a dose of about 1.0 mg about every 8 weeks (Q8W).

The amount of NGF antagonist contained within the individual doses may be expressed in terms of milligrams of antibody per kilogram of patient body weight (i.e., mg/kg). For example, the NGF antagonist may be administered to a patient at a dose of about 0.0001 to about 10 mg/kg of patient body weight. For example, the NGF antagonist may be administered to a patient at a dose of about 0.03 to about 3 mg/kg of patient body weight. For example, the NGF antagonist may be administered to a patient at a dose of about 0.03 to about 3 mg/kg of patient body weight.

### Combination Therapies

The methods of the present disclosure, according to certain embodiments, comprise administering to the subject one or more additional therapeutic agents in combination with the NGF antagonist. As used herein, the expression “in combination with” means that the additional therapeutic agents are administered before, after, or concurrent with the pharmaceutical composition comprising the NGF antagonist. The term “in combination with” also includes sequential or concomitant administration of NGF antagonist and a second therapeutic agent.

For example, when administered “before” the pharmaceutical composition comprising the NGF antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the NGF antagonist. When administered “after” the pharmaceutical composition comprising the NGF antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the NGF antagonist.

Administration “concurrent” or with the pharmaceutical composition comprising the NGF antagonist means that the additional therapeutic agent is administered to the subject in a separate dosage form within less than 5 minutes (before, after, or at the same time) of administration of the pharmaceutical composition comprising the NGF antagonist, or administered to the subject as a single combined dosage formulation comprising both the additional therapeutic agent and the NGF antagonist.

The additional therapeutic agent may be, e.g., another NGF antagonist (e.g. see the NGF antibodies described in U.S. Pat. No. 7,449,616 (tanezumab); U.S. Pat. Nos. 7,569,364; 7,655,232; 8,088,384; WO2011049758 (fulranumab)), an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), an opioid, acetaminophen, a local anesthestic, an NMDA modulator, a cannabinoid receptor agonist, a P2X family modulator, a VR1 antagonist, a substance P antagonist, a Naᵥ1.7 antagonist, a cytokine or cytokine receptor antagonist, an antiepileptic drug, a steroid, other inflammatory inhibitors such as inhibitors of caspase-1, p38, IKK1/2, CTLA-4lg and a corticosteroid.

In one embodiment, the additional therapeutic agent is not a NSAID. In some embodiments, the additional therapeutic agent excludes NSAIDs.

### Administration Regimens

The present disclosure includes methods comprising administering to a subject a pharmaceutical composition comprising an NGF antagonist at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments involving the administration of a pharmaceutical composition comprising an anti-NGF antibody, such as fasinumab, once every 4 weeks (Q4W) dosing at an amount of about 1 mg can be employed. In certain embodiments involving the administration of a pharmaceutical composition comprising an anti-NGF antibody, such as fasinumab, once every 8 weeks (Q8W) dosing at an amount of about 1 mg can be employed. In certain embodiments involving the administration of a pharmaceutical composition comprising an anti-NGF antibody, such as fasinumab, once every 12 weeks dosing at an amount of about 1 mg can be employed.

In some embodiments, the anti-NGF antibody, such as fasinumab, is administered at a dose of about 1.0 mg about every 4 weeks (Q4W). In some embodiments, the anti-NGF antibody, such as fasinumab, is administered at a dose of about 3.0 mg about every 4 weeks (Q4W). In some embodiments, the anti-NGF antibody, such as fasinumab, is administered at a dose of about 6.0 mg about every 4 weeks (Q4W). In some embodiments, the anti-NGF antibody, such as fasinumab, is administered at a dose of about 9.0 mg about every 4 weeks (Q4W). In some embodiments, the anti-NGF antibody, such as fasinumab, is administered at a dose of about 1.0 mg about every 8 weeks (Q8W).

According to certain embodiments of the present disclosure, multiple doses of an NGF antagonist may be administered to a subject over a defined time course. The methods according to this aspect of the disclosure comprise sequentially administering to a subject multiple doses of an NGF antagonist. As used herein, “sequentially administering” means that each dose of NGF antagonist is administered to the subject at a different point in time, e.g., on different days separated by a predetermined interval (e.g., hours, days, weeks or months). The present disclosure includes methods which comprise sequentially administering to the patient a single initial dose of an NGF antagonist, followed by one or more secondary doses of the NGF antagonist, and optionally followed by one or more tertiary doses of the NGF antagonist.

In some embodiments, the NGF antagonist is administered at one or more doses resulting in an improvement in one or more pain-associated parameters followed by the administration of a second therapeutic agent to prevent recurrence of at least one symptom of osteoarthritis pain. Alternative embodiments of the disclosure pertain to concomitant administration of an NGF antagonist and a second therapeutic agent. For example, one or more doses of an NGF antagonist are administered and a second therapeutic agent is administered at a separate dosage at a similar or different frequency relative to the NGF antagonist. In some embodiments, the second therapeutic agent is administered before, after or concurrently with the NGF antagonist.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Evaluation of Fasinumab for Treatment of Pain Associated with Osteoarthritis of the Knee or Hip

In a clinical study, the safety and tolerability of fasinumab was evaluated in patients with radiographically-confirmed OA of the knee or hip.

Patients: Eligible patients were 40-80 years of age; had a diagnosis of OA of the knee and/or hip (designated the most symptomatic, index joint at the time of the screening visit) based on the American College of Rheumatology criteria for OA with radiologic confirmation (Kellgren-Lawrence [K-L] grading of ≥2 on a scale of 0-4); and demonstrated moderate-to-severe pain in the index joint, defined as a Western Ontario and McMaster Universities Osteoarthritis Index (WOMAC) pain subscale score of ≥4, at both the screening while on usual analgesic medication and at the randomization visit, which occurred 7 days after withdrawal of analgesic therapy.

Study Design: Patients were randomized (1:1:1:1:1) to receive fasinumab 1 mg, 3 mg, 6 mg, or 9 mg or placebo administered subcutaneously every 4 weeks for a total of 4 doses over a 16-week treatment period. To ensure balanced treatment assignment across joints and OA severity, randomization was stratified by index joint (knee or hip) and K-L scores (2-3 vs 4). After the treatment period, patients were followed for an additional 20 weeks, resulting in a 36-week study period. Efficacy and safety assessments were performed at each study visit through week 36. Additional safety data were captured via telephone survey at weeks 24 and 32.

Endpoints: The primary efficacy endpoint was the change from baseline to week 16 in the WOMAC pain subscale (a composite index of 5 questions related to joint pain while walking, using stairs, at rest in bed, sitting or lying, and standing) (Bellamy N. London, Ontario, Canada: Victoria Hospital; 1995). Secondary efficacy endpoints were change from baseline to week 16 in the WOMAC physical function subscale score (scale of 0-10; Bellamy N. London, Ontario, Canada: Victoria Hospital; 1995) and Patient Global Assessment (PGA) score (a single question on a scale of 1-5, with worst assessment being the highest score (Strand V, Kellman A. Curr Rheumatol Rep 2004:6:20-30). Subjects were also assessed using the Short Form (36) Health Survey (SF-36), Medical Outcomes Study (MOS) sleep subscale, the EQ-5D-5L questionnaire, NRS average walking index joint pain, and actigraphy. Subjects were also assessed for centralized pain at week 1 using 2011 ACR fibromyalgia diagnostic criteria (Wolfe et al. (2011) J Rheumatol 38(6): 1113-22).

Results: A total of 1,214 patients were screened, 421 were randomized to receive fasinumab (n=338) or placebo (n=83). (see Table 2, below). Of the 421 randomized patients, 419 patients received ≥1 dose of study medication (1 patient each, randomized to placebo and fasinumab 9 mg, discontinued before study drug administration). A total of 342 patients completed the entire 36-week study period (fasinumab: n=294; 87%; placebo: n=67; 81%).

All four doses of fasinumab demonstrated significantly greater reductions from baseline at week 16 in WOMAC pain subscale scores than placebo. The LS mean difference over placebo for treatment groups ranged from −0.78 to −1.40, exceeding the published MCID (for an individual patient: 0.67-0.75 points), with the greatest difference observed with the 9-mg dose. Reductions in pain subscale scores were evident by week 2 across fasinumab doses and were maintained throughout the 16-week treatment period. During the follow-up period (after week 16), pain scores returned to baseline levels, though not fully, for each fasinumab dose.

Additionally, all 4 doses of fasinumab demonstrated statistically significant and clinically meaningful reductions from baseline at week 16 in WOMAC physical function subscale scores compared with placebo, with an incomplete return to baseline values, paralleling the changes noted for the WOMAC pain subscale. Across all doses, fasinumab was also associated with greater numerical reductions from baseline at week 16 in PGA scores than placebo, reaching statistical significance for the 1-mg and 9-mg doses (>30% improvement; P values: 0.0132 and 0.008, respectively). PGA scores returned to baseline levels in the follow-up period.

Conclusion: This clinical study involving >400 patients, the NGF-inhibitor fasinumab demonstrated an unprecedented degree of analgesia in patients with moderate-to-severe pain from OA, even in patients who had not experienced benefits with prior analgesic medications. Intensive laboratory and radiographic monitoring of patients during the trial demonstrated that fasinumab was well tolerated by most patients, with a dose-dependent increase in joint-related abnormalities notable at the two highest doses studied. For additional details on this clinical study, see U.S. Pub. No. 20200048337.

### Example 2: Evaluation of Fasinumab for Treatment of Pain Associated with OA in Subjects with High Centralized Pain Scores

To investigate the efficacy of fasinumab in patients with high levels of centralized pain, a supplementary analysis was performed on the data collected from the study summarized in Example 1.

For all doses of fasinumab and placebo, the 421 total subjects were grouped by their centralized pain score according to the 2011 ACR fibromyalgia diagnostic criteria described in Wolfe et al. (2011) J Rheumatol 38(6): 1113-22. The 2011 ACR fibromyalgia diagnostic criteria (QSPC) is depicted in FIG. 1.

The 2011 ACR fibromyalgia diagnostic criteria score (referred to herein as “FM score” or “QSPC score”) for each subject was collected on day 1 of the treatment period. Subjects were distributed into the following groups based on FM scores: 0-4 (low centralized pain), 5-8 (moderate centralized pain), and 9-31 (high centralized pain). See Table 2. This resulted in a distribution of 28% of subjects in the 0-4 (low) group, 28% of subjects in the 5-8 (moderate) group, and 30.4% of subjects in the 9-31 (high) group. The distribution of subjects is shown below in Table 2.

TABLE 2
Distribution of Subjects and FM Scores at Baseline

| | | | | | | |
|---|---|---|---|---|---|---|
| Placebo | 1 mg | 3 mg | 6 mg | 9 mg | Combined | Total |
| (N = 83) | (N = 85) | (N = 84) | (N = 85) | (N = 84) | (N = 338) | (N = 421) |

Total Score

| | | | | | | | |
|---|---|---|---|---|---|---|---|
| n | 71 | 74 | 72 | 74 | 73 | 293 | 364 |
| Mean (SD) | 7.1 (5.35) | 7.5 (4.83) | 7.3 (5.23) | 6.7 (4.45) | 7.8 (5.16) | 7.3 (4.92) | 7.3 (5.00) |
| Median | 6.0 | 7.0 | 6.0 | 6.0 | 7.0 | 7.0 | 7.0 |
| Q1:Q3 | 3:10 | 4:10 | 3:10 | 4:10 | 5:11 | 4:10 | 4:10 |
| Min:Max | 0:22 | 0:27 | 0:23 | 0:17 | 0:22 | 0:27 | 0:27 |

Total Score Category n (%)

| | | | | | | | |
|---|---|---|---|---|---|---|---|
| 0-4 | 29 (34.9%) | 20 (23.5%) | 24 (28.6%) | 27 (31.8%) | 18 (21.4%) | 89 (26.3%) | 118 (28.0%) |
| 5-8 | 18 (21.7%) | 25 (29.4%) | 26 (31.0%) | 19 (22.4%) | 30 (35.7%) | 100 (29.6%) | 118 (28.0%) |
| 9-31 | 24 (29.0%) | 29 (34.1%) | 22 (26.2%) | 28 (32.9%) | 25 (29.8%) | 104 (30.7%) | 128 (30.4%) |

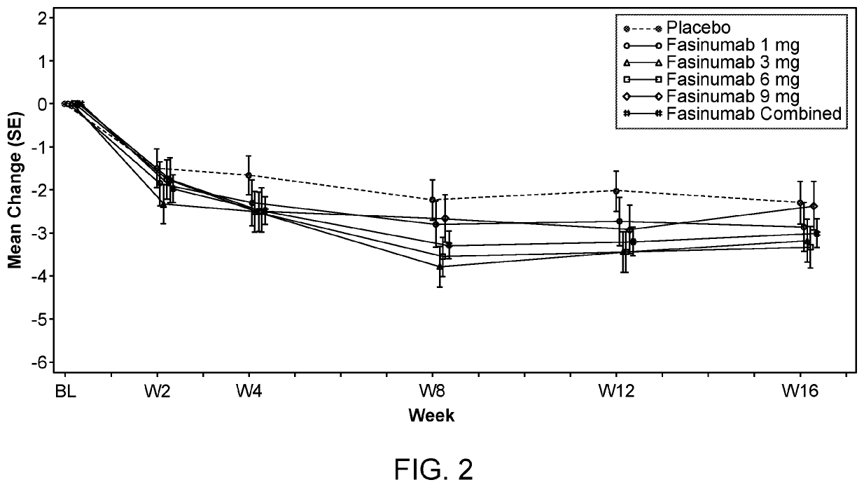

The change in WOMAC pain subscale scores over the treatment period of the study (16weeks) were analyzed for all subjects and distributed according to FM score group. Graphical representations of the results of this analysis are shown in FIGS. 2-6.

Results: Fasinumab-treated patients reporting FM scores of 0-4 reported only minor improvements in WOMAC pain subscale score when compared to patients receiving placebo. See FIG. 2 and Tables 3A-and 3B.

TABLE 3A
Change from Baseline in WOMAC Pain Subscale Score - FM Score 0-4 (mean SD)

| | Placebo | 1 mg | 3 mg | 6 mg | 9 mg | Combined |
|---|---|---|---|---|---|---|
| Baseline | 5.90 (1.961) | 6.11 (1.566) | 6.03 (1.634) | 6.12 (1.528) | 6.23 (1.352) | 6.12 (1.508) |
| Week 2 | −1.61 (1.426) | −2.16 (1.549) | −2.44 (2.042) | −1.93 (2.517) | −2.00 (2.047) | −2.13 (2.081) |
| Week 4 | −1.85 (1.678) | −2.63 (1.611) | −2.61 (2.148) | −2.82 (2.182) | −2.69 (1.963) | −2.69 (1.983) |
| Week 8 | −2.48 (2.127) | −3.10 (1.786) | −3.99 (2.179) | −3.84 (2.018) | −2.88 (2.488) | −3.51 (2.131) |
| Week 12 | −2.04 (1.978) | −3.03 (1.704) | −3.63 (2.205) | −3.62 (2.085) | −3.13 (2.392) | −3.38 (2.087) |
| Week 16 | −2.35 (2.446) | −3.20 (1.517) | −3.39 (2.195) | −3.60 (2.211) | −2.59 (2.507) | −3.24 (2.136) |

TABLE 3B
Change from Baseline in WOMAC Pain Subscale Score - FM Score 0-4 (mean LS (SE))

| | Placebo | 1 mg | 3 mg | 6 mg | 9 mg | Combined |
|---|---|---|---|---|---|---|
| Week 2 | −1.50 (0.449) | −1.86 (0.525) | −2.34 (0.454) | −1.75 (0.438) | −1.78 (0.526) | −1.97 (0.322) |
| Week 4 | −1.66 (0.448) | −2.30 (0.522) | −2.50 (0.447) | −2.53 (0.438) | −2.47 (0.518) | −2.49 (0.321) |
| Week 8 | −2.23 (0.469) | −2.80 (0.538) | −3.79 (0.470) | −3.55 (0.457) | −2.66 (0.540) | −3.28 (0.332) |
| Week 12 | −2.03 (0.480) | −2.73 (0.543) | −3.45 (0.476) | −3.45 (0.466) | −2.91 (0.546) | −3.20 (0.333) |
| Week 16 | −2.30 (0.496) | −2.86 (0.563) | −3.18 (0.496) | −3.33 (0.479) | −2.37 (0.564) | −3.01 (0.340) |

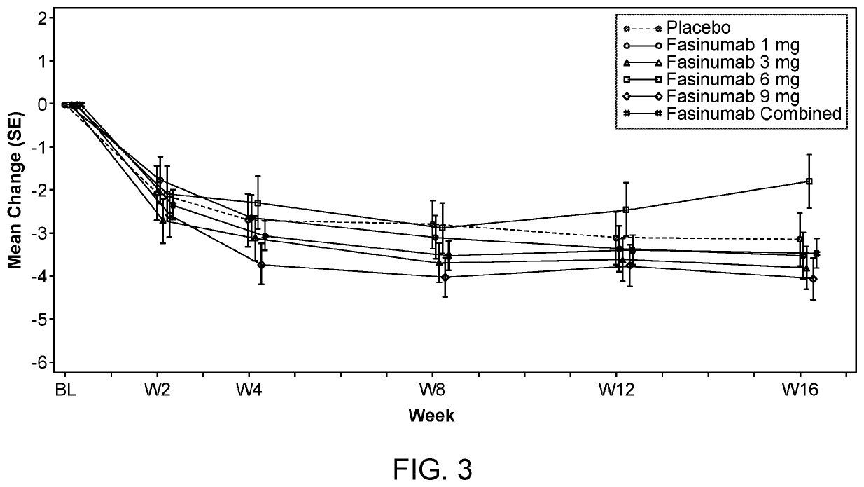

Fasinumab-treated patients reporting FM scores of 5-8 reported only minor improvements in WOMAC pain subscale score compared to patients receiving placebo. See FIG. 3 and Table 4A and 4B.

TABLE 4A
Change from Baseline in WOMAC Pain Subscale Score - FM Score 5-8 (mean SD)

| | Placebo | 1 mg | 3 mg | 6 mg | 9 mg | Combined |
|---|---|---|---|---|---|---|
| Baseline | 6.63 (1.383) | 6.55 (1.574) | 6.15 (1.653) | 5.85 (1.323) | 6.47 (1.524) | 6.29 (1.536) |
| Week 2 | −2.24 (2.423) | −2.27 (2.372) | −2.74 (2.161) | −2.14 (2.074) | −2.70 (2.787) | −2.50 (2.383) |
| Week 4 | −2.87 (2.027) | −2.98 (2.505) | −3.07 (2.679) | −2.29 (2.072) | −3.85 (2.334) | −3.14 (2.453) |
| Week 8 | −2.97 (2.117) | −3.62 (2.169) | −3.65 (2.295) | −2.90 (2.185) | −4.15 (2.127) | −3.66 (2.201) |
| Week 12 | −3.28 (2.184) | −4.02 (2.307) | −3.65 (2.685) | −2.48 (2.274) | −3.89 (2.396) | −3.58 (2.460) |
| Week 16 | −3.45 (1.983) | −4.15 (2.381) | −3.77 (2.725) | −2.01 (2.224) | −4.19 (2.448) | −3.67 (2.565) |

TABLE 4B
Change from Baseline in WOMAC Pain Subscale Score - FM Score 5-8 (mean LS (SE))

| | Placebo | 1 mg | 3 mg | 6 mg | 9 mg | Combined |
|---|---|---|---|---|---|---|
| Week 2 | −2.05 (0.633) | −1.75 (0.548) | −2.69 (0.523) | −2.06 (0.639) | −2.56 (0.508) | −2.32 (0.350) |
| Week 4 | −2.67 (0.608) | −2.61 (0.522) | −3.08 (0.499) | −2.27 (0.620) | −3.70 (0.490) | −3.03 (0.344) |
| Week 8 | −2.77 (0.561) | −3.07 (0.490) | −3.66 (0.459) | −2.86 (0.576) | −4.00 (0.454) | −3.50 (0.329) |
| Week 12 | −3.08 (0.605) | −3.33 (0.530) | −3.58 (0.498) | −2.44 (0.618) | −3.74 (0.487) | −3.37 (0.344) |
| Week 16 | −3.12 (0.609) | −3.50 (0.535) | −3.78 (0.497) | −1.78 (0.623) | −4.04 (0.487) | −3.44 (0.350) |

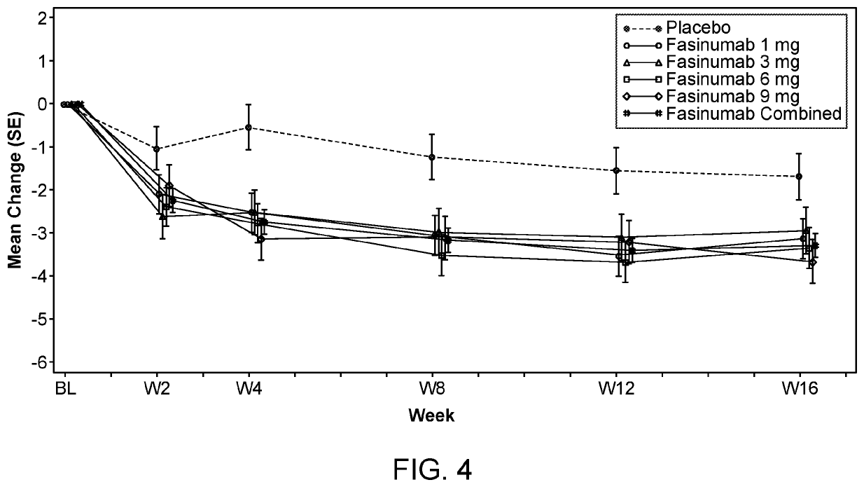

Fasinumab-treated subjects reporting FM scores of 9-31 showed a much more significant improvement in WOMAC pain subscale scores when compared to subjects receiving placebo. See FIG. 4 and Tables 5A-5B.

TABLE 5A
Change from Baseline in WOMAC Pain Subscale Score - FM Score 9-31 (mean SD)

| | Placebo | 1 mg | 3 mg | 6 mg | 9 mg | Combined |
|---|---|---|---|---|---|---|
| Baseline | 7.10 (1.389) | 6.69 (1.595) | 6.73 (1.520) | 6.31 (1.436) | 7.10 (1.468) | 6.69 (1.512) |
| Week 2 | −1.25 (2.048) | −2.16 (2.172) | −2.73 (2.161) | −2.31 (2.444) | −2.09 (2.388) | −2.30 (2.277) |
| Week 4 | −0.77 (1.808) | −2.59 (2.296) | −2.70 (1.964) | −2.69 (2.416) | −3.34 (2.518) | −2.82 (2.309) |
| Week 8 | −1.65 (2.375) | −3.17 (2.121) | −3.13 (2.403) | −3.50 (2.280) | −3.30 (2.542) | −3.28 (2.301) |
| Week 12 | −2.03 (2.570) | −3.68 (2.451) | −3.29 (2.134) | −3.58 (2.093) | −3.50 (2.622) | −3.53 (2.308) |
| Week 16 | −2.11 (2.508) | −3.26 (2.128) | −3.08 (2.348) | −3.27 (2.356) | −4.03 (2.596) | −3.41 (2.341) |

TABLE 5B
Change from Baseline in WOMAC Pain Subscale Score - FM Score 9-31 (mean LS (SE))

| | Placebo | 1 mg | 3 mg | 6 mg | 9 mg | Combined |
|---|---|---|---|---|---|---|
| Week 2 | −1.03 (0.511) | −2.09 (0.446) | −2.62 (0.521) | −2.39 (0.450) | −1.89 (0.487) | −2.24 (0.272) |
| Week 4 | −0.54 (0.507) | −2.52 (0.442) | −2.51 (0.520) | −2.76 (0.446) | −3.14 (0.483) | −2.74 (0.270) |
| Week 8 | −1.22 (0.537) | −3.05 (0.464) | −2.97 (0.543) | −3.52 (0.469) | −3.09 (0.502) | −3.17 (0.279) |
| Week 12 | −1.54 (0.538) | −3.52 (0.467) | −3.10 (0.543) | −3.68 (0.472) | −3.21 (0.507) | −3.40 (0.281) |
| Week 16 | −1.68 (0.533) | −3.13 (0.467) | −2.94 (0.543) | −3.34 (0.472) | −3.66 (0.510) | −3.28 (0.282) |

Conclusion: Patients reporting higher centralized pain scores (e.g. FM scores) have been correlated with poorer pain improvement outcomes (Brummett 2015). However, the results of this study show that subjects reporting higher centralized pain scores (e.g. FM scores) when starting fasinumab treatment also reported a substantial reduction in OA joint pain with fasinumab treatment. These results did not appear to be dependent on the dose of fasinumab received by the subject.

Therefore, it appears that fasinumab treatment may provide substantial pain improvement in patients with high levels of centralized pain.

### Example 3: Evaluation of Fasinumab as Treatment for Centralized Pain

Subjects reporting higher centralized pain scores from the 2011 ACR fibromyalgia diagnostic criteria (“FM scores”) also reported substantial reductions in WOMAC pain subscale score with fasinumab treatment (see Example 2). However, the WOMAC pain subscale score is designed to evaluate only one type of pain (i.e., joint pain in the index joint only). To evaluate the potential efficacy of fasinumab in reducing centralized pain, progression of subjects' generalized pain scores over the treatment period was analyzed. The scores of selected questions from the SF-36 and EQ-5D-5L surveys collected during the clinical trial were selected as generalized pain scoresand tracked over time.

On study day 1, centralized pain scores (FM scores) were collected from all subjects. However, FM score was not collected at later time points throughout the treatment period and thus was unavailable to evaluate central pain progression. Instead, centralized pain progression was analyzed using changes in scores from selected questions from the SF-36 and EQ5D survey forms as “generalized pain scores”, acting as proxies for more conventional centralized pain scores (e.g. FM score). During the treatment period, subjects completed the SF-36 and EQ5D surveys on study day 1, as well as week 1, 2, 3, 8, 12, and 16.

Item 7 and item 8 of the SF-36 survey, which relate to bodily pain and pain interference with work, respectively, were selected as generalized pain scores. Subjects were distributed into the following groups based on pre-treatment FM scores: 0-4 (low centralized pain), 5-8 (moderate centralized pain), and 9-31 (high centralized pain). See Table 2. Reported scores in SF-36 items 7 and 8 were analyzed for each group of subjects over the treatment period of the study (16 weeks)

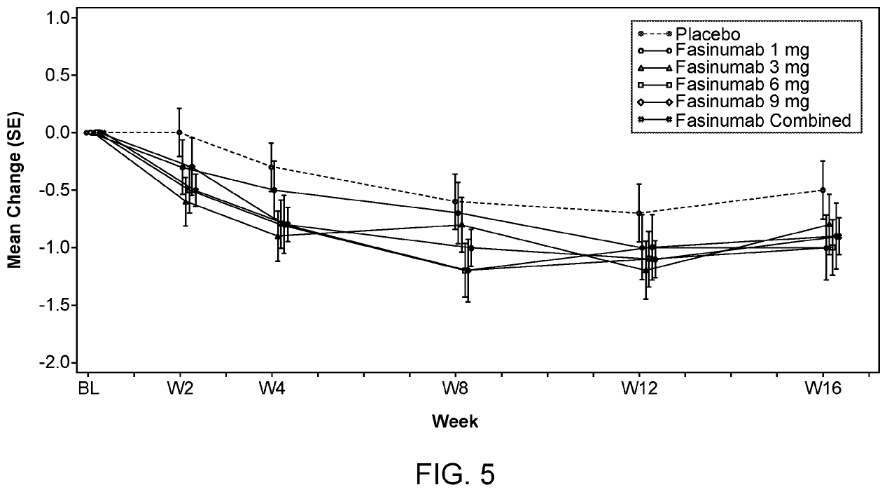

Results: SF-36 item 7 inquires: “How much bodily pain have you had during the past 4 weeks?” and records a score from 1-6. Changes in SF-36 item 7 scores over time are shown in FIGS. 5-7. Subjects receiving fasinumab in the low centralized pain group did not show a significantly different change in SF-36 item 7 scores when compared to placebo. See FIG. 5 and Tables 6A-6B.

TABLE 6A
Change from Baseline in SF36-Bodily Pain (Item 7) Score - FM Score 0-4 (mean (SD))

| | Placebo | 1 mg | 3 mg | 6 mg | 9 mg | Combined |
|---|---|---|---|---|---|---|
| Baseline | 3.9 (0.76) | 3.4 (0.52) | 2.9 (0.92) | 3.6 (0.53) | 3.6 (0.92) | 3.3 (0.83) |
| Week 2 | −0.5 (0.97) | −0.6 (1.06) | −0.1 (0.83) | −0.6 (1.13) | −0.6 (1.21) | −0.4 (1.05) |
| Week 4 | −0.4 (1.12) | −0.8 (1.28) | 0.0 (0.91) | −0.6 (1.13) | −1.1 (1.38) | −0.6 (1.21) |
| Week 8 | −0.8 (1.22) | −0.6 (1.06) | −0.2 (0.99) | −0.7 (1.11) | −1.2 (1.40) | −0.6 (1.18) |
| Week 12 | −0.8 (1.06) | −0.9 (1.25) | −0.3 (0.85) | −0.7 (1.11) | −1.6 (1.35) | −0.8 (1.20) |
| Week 16 | −0.7 (1.15) | −0.1 (1.57) | 0.2 (1.07) | −0.7 (0.95) | −1.6 (1.07) | −0.5 (1.32) |

TABLE 6B
Change from Baseline in SF36-Bodily Pain (Item 7) Score - FM Score 0-4 (mean LS (SE))

| | Placebo | 1 mg | 3 mg | 6 mg | 9 mg | Combined |
|---|---|---|---|---|---|---|
| Week 2 | 0.1 (0.30) | −0.5 (0.34) | −0.1 (0.29) | −0.4 (0.35) | −0.3 (0.31) | −0.3 (0.18) |
| Week 4 | −0.2 (0.29) | −0.7 (0.33) | −0.3 (0.29) | −0.4 (0.34) | −0.7 (0.31) | −0.5 (0.17) |
| Week 8 | −0.1 (0.30) | −0.5 (0.33) | −0.4 (0.29) | −0.6 (0.34) | −0.8 (0.31) | −0.6 (0.17) |
| Week 12 | 0.0 (0.31) | −0.8 (0.34) | −0.6 (0.30) | −0.6 (0.35) | −1.1 (0.32) | −0.8 (0.18) |
| Week 16 | 0.0 (0.32) | 0.0 (0.37) | −0.1 (0.31) | −0.6 (0.36) | −1.1 (0.33) | −0.5 (0.20) |

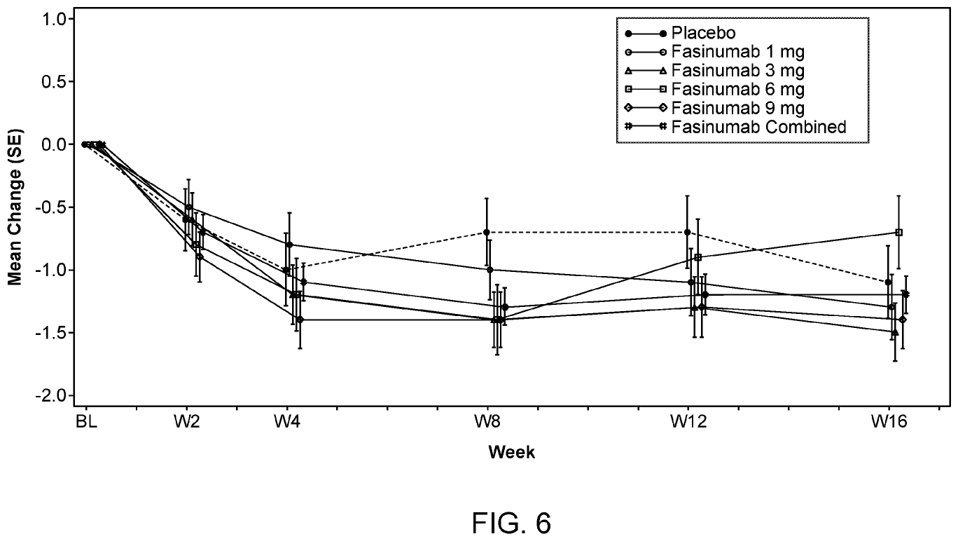

Subjects in the moderate centralized pain group who received fasinumab did not show a significantly different change in SF-36 item 7 scores when compared to placebo. See FIG. 6 and Tables 7A-7B.

TABLE 7A
Change from Baseline in SF36-Bodily Pain (Item 7) Score - FM Score 5-8 (mean (SD))

| | Placebo | 1 mg | 3 mg | 6 mg | 9 mg | Combined |
|---|---|---|---|---|---|---|
| Baseline | 4.1 (0.42) | 4.2 (0.58) | 4.2 (0.67) | 4.1 (0.66) | 4.3 (0.60) | 4.2 (0.62) |
| Week 2 | −0.9 (1.23) | −0.8 (1.05) | −1.1 (1.18) | −1.2 (1.10) | −1.4 (1.30) | −1.2 (1.18) |
| Week 4 | −0.3 (1.44) | −0.7 (1.05) | −1.3 (1.23) | −0.9 (0.95) | −0.5 (1.58) | −0.9 (1.21) |
| Week 8 | −0.6 (0.98) | −1.1 (1.06) | −1.3 (1.16) | −1.3 (1.08) | −1.5 (1.17) | −1.3 (1.12) |
| Week 12 | −0.6 (0.85) | −1.3 (0.85) | −1.3 (1.57) | −0.8 (0.92) | −1.4 (1.35) | −1.2 (1.25) |
| Week 16 | −0.9 (0.75) | −1.5 (0.98) | −1.4 (1.60) | −0.7 (0.69) | −1.4 (1.22) | −1.3 (1.23) |

TABLE 7B
Change from Baseline in SF36-Bodily Pain (Item 7) Score - FM Score 5-8 (mean LS (SE))

| | Placebo | 1 mg | 3 mg | 6 mg | 9 mg | Combined |
|---|---|---|---|---|---|---|
| Week 2 | −0.6 (0.25) | −0.5 (0.22) | −0.6 (0.21) | −0.8 (0.25) | −0.9 (0.20) | −0.7 (0.14) |
| Week 4 | −1.0 (0.29) | −0.8 (0.25) | −1.2 (0.24) | −1.2 (0.29) | −1.4 (0.23) | −1.1 (0.15) |
| Week 8 | −0.7 (0.27) | −1.0 (0.24) | −1.4 (0.22) | −1.4 (0.28) | −1.4 (0.22) | −1.3 (0.15) |
| Week 12 | −0.7 (0.29) | −1.1 (0.27) | −1.3 (0.24) | −0.9 (0.30) | −1.3 (0.24) | −1.2 (0.16) |
| Week 16 | −1.1 (0.29) | −1.3 (0.26) | −1.5 (0.23) | −0.7 (0.29) | −1.4 (0.23) | −1.2 (0.15) |

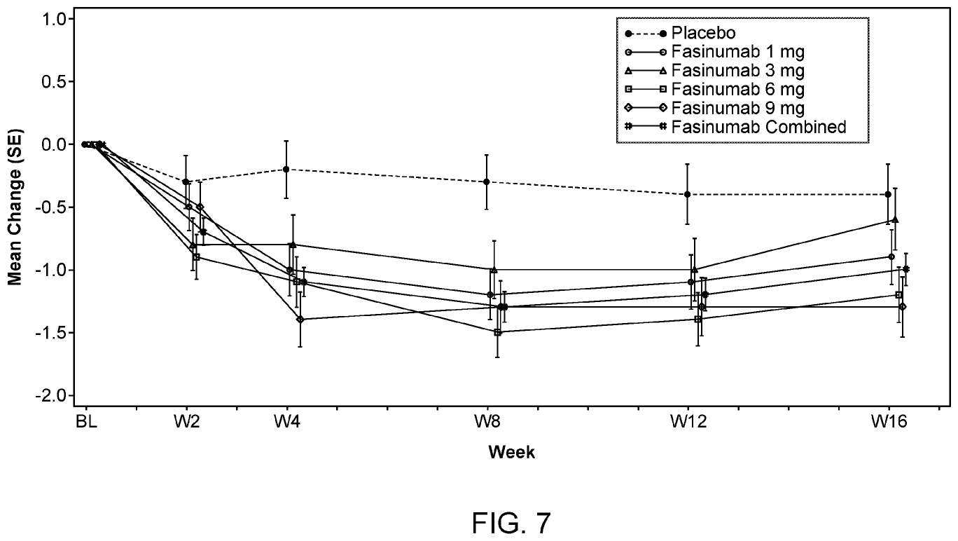

Subjects in the high centralized pain group who received fasinumab showed an enhanced reduction in SF-36 item 7 scores over time when compared to placebo. See FIG. 7 and Tables 8A-8B. The reduction appeared more pronounced in subjects receiving the 6 mg or 9 mg dose of fasinumab (FIG. 7, open squares and open diamonds, respectively).

TABLE 8A
Change from Baseline in SF36-Bodily Pain (Item 7) Score - FM Score 9-31 (mean (SD))

| | Placebo | 1 mg | 3 mg | 6 mg | 9 mg | Combined |
|---|---|---|---|---|---|---|
| Baseline | 4.7 (0.76) | 4.6 (0.56) | 4.4 (0.73) | 4.4 (0.68) | 4.8 (0.78) | 4.5 (0.70) |
| Week 2 | −0.4 (0.82) | −0.6 (0.99) | −0.8 (1.07) | −0.8 (1.03) | −0.6 (0.86) | −0.7 (0.98) |
| Week 4 | −0.3 (0.94) | −1.0 (1.00) | −0.8 (1.03) | −1.0 (1.09) | −1.5 (1.42) | −1.1 (1.16) |
| Week 8 | −0.4 (1.14) | −1.2 (1.12) | −1.0 (1.02) | −1.4 (0.97) | −1.4 (1.12) | −1.3 (1.06) |
| Week 12 | −0.5 (1.03) | −1.2 (1.36) | −1.0 (0.89) | −1.2 (1.14) | −1.5 (1.25) | −1.2 (1.18) |
| Week 16 | −0.5 (1.01) | −1.0 (1.27) | −0.6 (1.31) | −1.1 (1.01) | −1.5 (1.44) | −1.1 (1.28) |

TABLE 8B
SF36 Item 7 scores for subjects with centralized pain scores (9-31) (mean LS (SE))

| | Placebo | 1 mg | 3 mg | 6 mg | 9 mg | Combined |
|---|---|---|---|---|---|---|
| Week 2 | −0.3 (0.21) | −0.5 (0.19) | −0.8 (0.21) | −0.9 (0.18) | −0.5 (0.20) | −0.7 (0.11) |
| Week 4 | −0.2 (0.23) | −1.0 (0.21) | −0.8 (0.24) | −1.1 (0.20) | −1.4 (0.22) | −1.1 (0.12) |
| Week 8 | −0.3 (0.23) | −1.2 (0.20) | −1.0 (0.23) | −1.5 (0.20) | −1.3 (0.21) | −1.3 (0.12) |
| Week 12 | −0.4 (0.24) | −1.1 (0.22) | −1.0 (0.25) | −1.4 (0.21) | −1.3 (0.23) | −1.2 (0.13) |
| Week 16 | −0.4 (0.24) | −0.9 (0.22) | −0.6 (0.25) | −1.2 (0.22) | −1.3 (0.24) | −1.0 (0.13) |

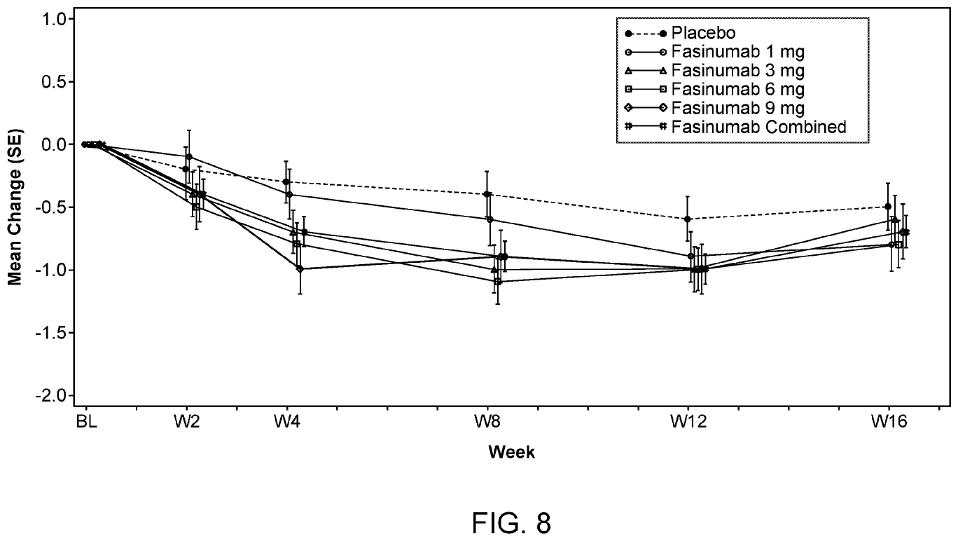

Results: SF-36 item 8 inquires: “During the past 4 weeks, how much did pain interfere with your normal work (including both work outside the home and housework)?” and records a score from 1-5. Changes in SF-36 item 8 over time are shown in FIGS. 8-10. Subjects in the low centralized pain group who received fasinumab did not show a significantly different change in SF-36 item 8 scores when compared to placebo. See FIG. 8 and Tables 9A-9B.

TABLE 9A
Change from Baseline in SF36-Pain Interference
(Item 8) Score - FM Score 0-4 (mean (SD))

| | Placebo | 1 mg | 3 mg | 6 mg | 9 mg | Combined |
|---|---|---|---|---|---|---|
| Baseline | 2.9 (0.99) | 3.3 (1.08) | 3.2 (0.83) | 3.0 (0.98) | 2.6 (1.04) | 3.1 (0.99) |
| Week 2 | −0.3 (1.16) | −0.4 (0.99) | −0.7 (0.64) | −0.6 (1.01) | −0.3 (1.07) | −0.5 (0.93) |
| Week 4 | −0.3 (1.06) | −1.0 (1.23) | −1.2 (0.90) | −1.3 (1.08) | −0.7 (0.83) | −1.1 (1.03) |
| Week 8 | −0.4 (1.04) | −0.9 (0.97) | −1.1 (0.85) | −1.1 (0.99) | −0.6 (0.85) | −1.0 (0.93) |
| Week 12 | −0.6 (0.88) | −1.3 (1.07) | −1.2 (0.85) | −1.1 (1.19) | −0.9 (1.02) | −1.1 (1.03) |
| Week 16 | −0.5 (1.22) | −1.1 (1.08) | −0.9 (1.02) | −0.9 (1.01) | −0.6 (0.78) | −0.9 (0.98) |

TABLE 9B
Change from Baseline in SF36-Pain Interference
(Item 8) Score - FM Score 0-4 (mean LS (SE))

| | Placebo | 1 mg | 3 mg | 6 mg | 9 mg | Combined |
|---|---|---|---|---|---|---|
| Week 2 | −0.2 (0.18) | −0.1 (0.21) | −0.4 (0.18) | −0.5 (0.18) | −0.4 (0.22) | −0.4 (0.12) |
| Week 4 | −0.3 (0.17) | −0.4 (0.20) | −0.7 (0.17) | −0.8 (0.17) | −1.0 (0.20) | −0.7 (0.12) |
| Week 8 | −0.4 (0.18) | −0.6 (0.21) | −1.0 (0.19) | −1.1 (0.18) | −0.9 (0.21) | −0.9 (0.12) |
| Week 12 | −0.6 (0.18) | −0.9 (0.20) | −1.0 (0.18) | −1.0 (0.17) | −1.0 (0.20) | −1.0 (0.12) |
| Week 16 | −0.5 (0.19) | −0.8 (0.22) | −0.6 (0.19) | −0.8 (0.19) | −0.7 (0.22) | −0.7 (0.13) |

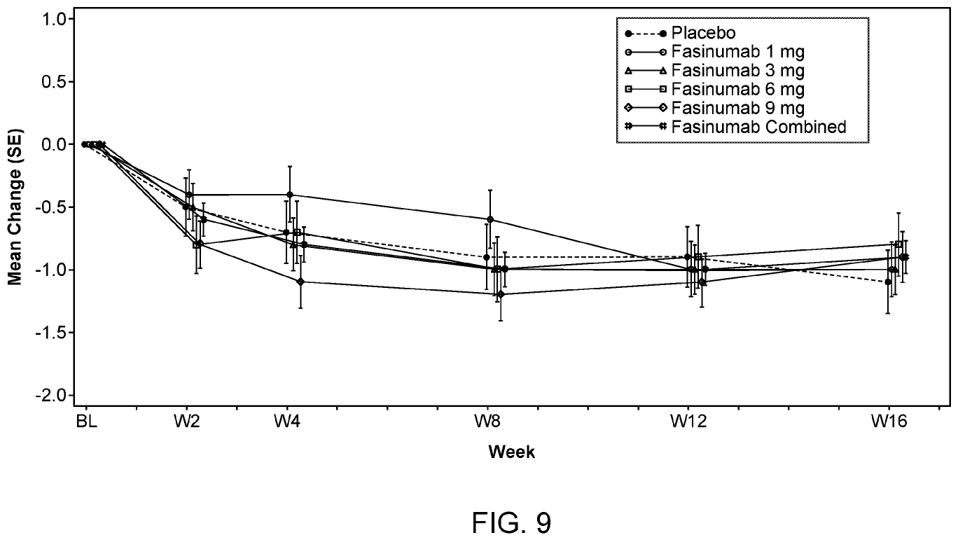

Subjects in the moderate centralized pain group who received fasinumab did not show a significantly different change in SF-36 item 8 scores when compared to placebo. See FIG. 9 and Tables 10A-10B.

TABLE 10A
Change from Baseline in SF36-Pain Interference (Item 8) Score - FM Score 5-8 (mean (SD))

| | Placebo | 1 mg | 3 mg | 6 mg | 9 mg | Combined |
|---|---|---|---|---|---|---|
| Baseline | 3.1 (0.73) | 3.1 (0.91) | 2.8 (0.86) | 2.6 (1.02) | 3.4 (0.94) | 3.0 (0.97) |
| (mean (SD)) | | | | | | |
| Week 2 | −0.4 (1.04) | −0.4 (0.84) | −0.4 (0.76) | −0.5 (1.22) | −0.9 (1.06) | −0.6 (0.99) |
| Week 4 | −0.6 (1.14) | −0.4 (0.97) | −0.6 (1.13) | −0.4 (1.09) | −1.2 (1.07) | −0.7 (1.11) |
| Week 8 | −0.8 (0.94) | −0.7 (0.77) | −0.8 (1.24) | −0.6 (1.09) | −1.3 (1.20) | −0.9 (1.13) |
| Week 12 | −0.8 (0.94) | −1.0 (0.80) | −0.8 (1.42) | −0.6 (0.78) | −1.2 (1.03) | −0.9 (1.08) |
| Week 16 | −1.1 (0.75) | −1.0 (0.80) | −0.8 (1.31) | −0.5 (1.18) | −1.0 (1.03) | −0.9 (1.10) |

TABLE 10B
Change from Baseline in SF36-Pain Interference (Item 8) Score - FM Score 5-8 (mean LS (SE))

| | Placebo | 1 mg | 3 mg | 6 mg | 9 mg | Combined |
|---|---|---|---|---|---|---|
| Week 2 | −0.5 (0.23) | −0.4 (0.20) | −0.5 (0.19) | −0.8 (0.23) | −0.8 (0.19) | −0.6 (0.13) |
| (mean LS (SE)) | | | | | | |
| Week 4 | −0.7 (0.25) | −0.4 (0.22) | −0.8 (0.21) | −0.7 (0.25) | −1.1 (0.21) | −0.8 (0.14) |
| Week 8 | −0.9 (0.26) | −0.6 (0.23) | −1.0 (0.21) | −1.0 (0.26) | −1.2 (0.21) | −1.0 (0.14) |
| Week 12 | −0.9 (0.24) | −1.0 (0.22) | −1.0 (0.20) | −0.9 (0.25) | −1.1 (0.20) | −1.0 (0.13) |
| Week 16 | −1.1 (0.25) | −1.0 (0.22) | −1.0 (0.20) | −0.8 (0.25) | −0.9 (0.20) | −0.9 (0.13) |

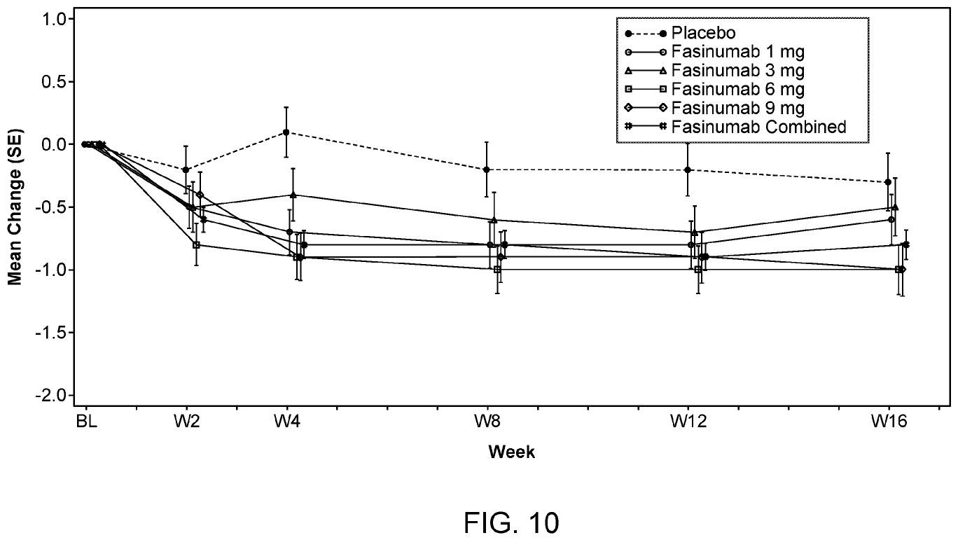

Subjects in the high centralized pain group who received fasinumab showed an enhanced reduction in SF-36 item 8 scores when compared to placebo (see FIG. 10 and Tables 11A-11B). Similar to the result for SF-36 item 7, the reduction appeared more pronounced in subjects receiving the 6 mg or 9 mg dose of fasinumab (FIG. 10, open squares and open diamonds, respectively).

TABLE 11A
Change from Baseline in SF36-Pain Interference
(Item 8) Score - FM Score 9-31 (mean (SD))

| | Placebo | 1 mg | 3 mg | 6 mg | 9 mg | Combined |
|---|---|---|---|---|---|---|
| Baseline | 3.6 (0.77) | 3.3 (0.93) | 3.3 (1.13) | 3.3 (0.77) | 3.6 (0.92) | 3.4 (0.93) |
| Week 2 | −0.3 (0.64) | −0.5 (1.07) | −0.5 (0.80) | −0.8 (0.80) | −0.5 (1.08) | −0.6 (0.95) |
| Week 4 | −0.1 (0.93) | −0.7 (0.98) | −0.4 (0.92) | −0.9 (0.60) | −1.0 (1.24) | −0.8 (0.97) |
| Week 8 | −0.4 (0.85) | −0.7 (1.02) | −0.6 (0.98) | −1.0 (1.09) | −1.0 (1.15) | −0.9 (1.07) |
| Week 12 | −0.4 (0.80) | −0.8 (0.97) | −0.8 (0.83) | −1.0 (0.94) | −1.0 (1.29) | −0.9 (1.01) |
| Week 16 | −0.5 (0.91) | −0.6 (1.08) | −0.5 (1.28) | −1.0 (0.96) | −1.2 (1.30) | −0.8 (1.17) |

TABLE 11B
Change from Baseline in SF36-Pain Interference
(Item 8) Score - FM Score 9-31 (mean LS (SE))

| | Placebo | 1 mg | 3 mg | 6 mg | 9 mg | Combined |
|---|---|---|---|---|---|---|
| Week 2 | −0.2 (0.19) | −0.5 (0.17) | −0.5 (0.20) | −0.8 (0.17) | −0.4 (0.18) | −0.6 (0.10) |
| Week 4 | 0.1 (0.20) | −0.7 (0.18) | −0.4 (0.21) | −0.9 (0.18) | −0.9 (0.19) | −0.8 (0.11) |
| Week 8 | −0.2 (0.22) | −0.8 (0.19) | −0.6 (0.22) | −1.0 (0.19) | −0.9 (0.20) | −0.8 (0.11) |
| Week 12 | −0.2 (0.21) | −0.8 (0.19) | −0.7 (0.21) | −1.0 (0.19) | −0.9 (0.20) | −0.9 (0.11) |
| Week 16 | −0.3 (0.23) | −0.6 (0.20) | −0.5 (0.23) | −1.0 (0.20) | −1.0 (0.21) | −0.8 (0.12) |

Based on the results of the SF-36 item 7 and item 8 analysis, it appears that fasinumab treatment may provide generalized pain relief in subjects reporting high levels of centralized pain at baseline.

Item 4 of the EQ-5D-5L survey, which relates to non-specific pain/discomfort, was selected as a generalized pain score. Scores ranged from 1 to 5 based on selection of the respective box (none, slight, moderate, severe, or extreme pain/discomfort). As above, subjects were distributed into the following groups based on pre-treatment FM scores: 0-4 (low centralized pain), 5-8 (moderate centralized pain), and 9-31 (high centralized pain). See Table 2. Reported scores for item 4 of the EQ5D over the fasinumab treatment period (16 weeks) were analyzed for each group.

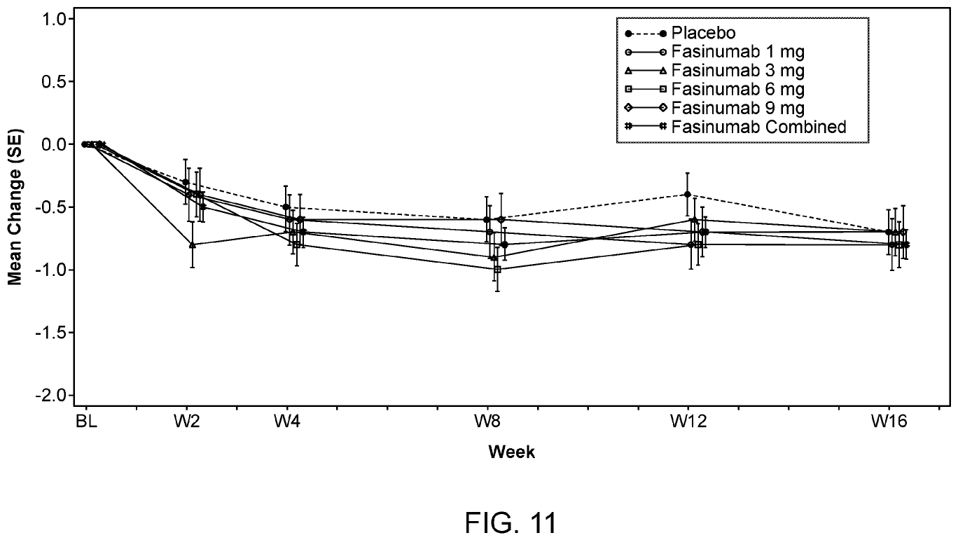

Results: Changes in EQ-5D-5L item 4 over time are shown in FIGS. 11-13. Subjects in the low centralized pain group who received fasinumab did not show a significantly different change in EQ5D item 4 scores when compared to placebo. See FIG. 11, Tables 12A-12B.

TABLE 12A
Change from Baseline in EQ5D-Pain/Discomfort
(Item 4) Score - FM Score 0-4 (mean (SD))

| | Placebo | 1 mg | 3 mg | 6 mg | 9 mg | Combined |
|---|---|---|---|---|---|---|
| Baseline | 3.0 (0.78) | 3.3 (0.97) | 3.2 (0.51) | 3.1 (0.78) | 2.9 (0.83) | 3.1 (0.78) |
| Week 2 | −0.3 (1.01) | −0.6 (0.82) | −1.0 (0.93) | −0.4 (0.90) | −0.3 (0.84) | −0.6 (0.90) |
| Week 4 | −0.5 (1.07) | −0.7 (0.87) | −0.9 (0.80) | −0.9 (0.82) | −0.6 (0.98) | −0.8 (0.85) |
| Week 8 | −0.6 (1.08) | −0.9 (0.97) | −1.1 (0.85) | −1.1 (0.99) | −0.6 (0.85) | −1.0 (0.93) |
| Week 12 | −0.4 (0.93) | −1.0 (0.92) | −0.8 (0.80) | −0.9 (1.00) | −0.7 (0.84) | −0.8 (0.89) |
| Week 16 | −0.7 (1.23) | −0.9 (0.91) | −0.9 (0.92) | −0.9 (1.03) | −0.7 (0.59) | −0.9 (0.89) |

TABLE 12B
Change from Baseline in EQ5D-Pain/Discomfort
(Item 4) Score - FM Score 0-4 (mean LS (SE))

| | Placebo | 1 mg | 3 mg | 6 mg | 9 mg | Combined |
|---|---|---|---|---|---|---|
| Week 2 | −0.3 (0.18) | −0.4 (0.21) | −0.8 (0.18) | −0.4 (0.18) | −0.4 (0.21) | −0.5 (0.12) |
| Week 4 | −0.5 (0.17) | −0.6 (0.20) | −0.7 (0.17) | −0.8 (0.17) | −0.6 (0.20) | −0.7 (0.12) |
| Week 8 | −0.6 (0.18) | −0.7 (0.21) | −0.9 (0.19) | −1.0 (0.18) | −0.6 (0.21) | −0.8 (0.18) |
| Week 12 | −0.4 (0.17) | −0.8 (0.20) | −0.6 (0.17) | −0.8 (0.17) | −0.7 (0.20) | −0.7 (0.0) |
| Week 16 | −0.7 (0.18) | −0.8 (0.21) | −0.7 (0.19) | −0.8 (0.18) | −0.7 (0.21) | −0.8 (0.12) |

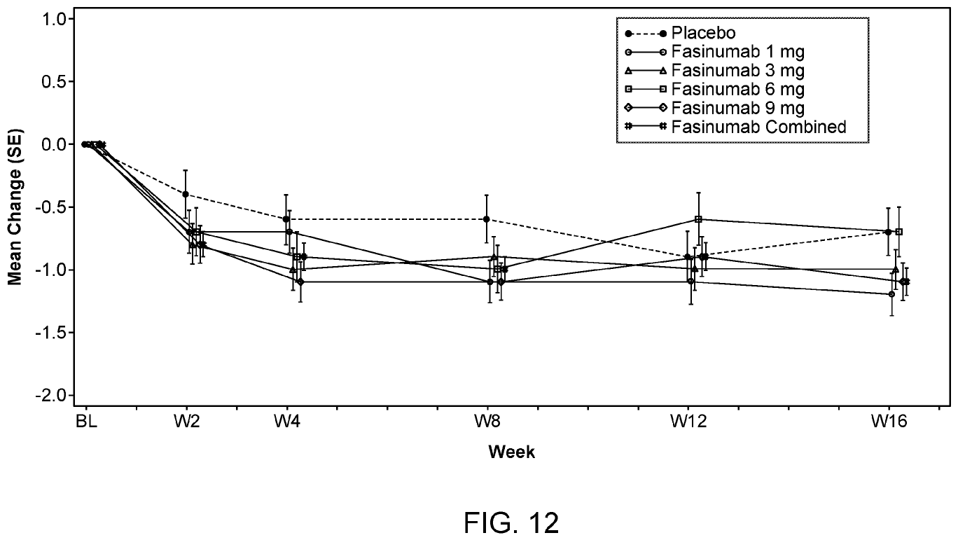

Subjects in the moderate centralized pain group who received fasinumab did not show a significantly different change in EQ-5D-5L item 4 scores when compared to placebo. See FIG. 12 and Tables 13A-13B.

TABLE 13A
Change from Baseline in EQ5D-Pain/Discomfort
(Item 4) Score - FM Score 5-8 (mean (SD))

| | Placebo | 1 mg | 3 mg | 6 mg | 9 mg | Combined |
|---|---|---|---|---|---|---|
| Baseline | 3.0 (0.49) | 3.4 (0.57) | 3.1 (0.56) | 3.1 (0.66) | 3.3 (0.48) | 3.2 (0.57) |
| Week 2 | −0.3 (0.67) | −0.8 (0.72) | −0.8 (0.71) | −0.7 (0.89) | −0.8 (0.76) | −0.8 (0.75) |
| Week 4 | −0.5 (0.86) | −0.8 (0.68) | −0.9 (0.89) | −0.8 (0.79) | −1.1 (0.82) | −0.9 (0.80) |
| Week 8 | −0.5 (0.71) | −1.2 (0.75) | −0.8 (0.73) | −0.9 (0.80) | −1.1 (0.15) | −1.0 (0.75) |
| Week 12 | −0.8 (0.81) | −1.2 (0.62) | −0.9 (0.91) | −0.6 (0.92) | −0.9 (0.16) | −0.9 (0.11) |
| Week 16 | −0.6 (0.61) | −1.4 (0.80) | −1.0 (0.87) | −0.8 (0.75) | −1.1 (0.73) | −1.1 (0.81) |

TABLE 13B
Change from Baseline in EQ5D-Pain/Discomfort
(Item 4) Score - FM Score 5-8 (mean LS (SE))

| | Placebo | 1 mg | 3 mg | 6 mg | 9 mg | Combined |
|---|---|---|---|---|---|---|
| Week 2 | −0.4 (0.19) | −0.7 (0.17) | −0.8 (0.16) | −0.7 (0.19) | −0.8 (0.15) | −0.8 (0.10) |
| Week 4 | −0.6 (0.20) | −0.7 (0.17) | −1.0 (0.17) | −0.9 (0.20) | −1.1 (0.16) | −0.9 (0.20) |
| Week 8 | −0.6 (0.19) | −1.1 (0.17) | −0.9 (0.16) | −1.0 (0.19) | −1.1 (0.15) | −1.0 (0.19) |
| Week 12 | −0.9 (0.20) | −1.1 (0.18) | −1.0 (0.17) | −0.6 (0.21) | −0.9 (0.16) | −0.9 (0.11) |
| Week 16 | −0.7 (0.19) | −1.2 (0.17) | −1.0 (0.16) | −0.7 (0.20) | −1.1 (0.15) | −1.1 (0.11) |

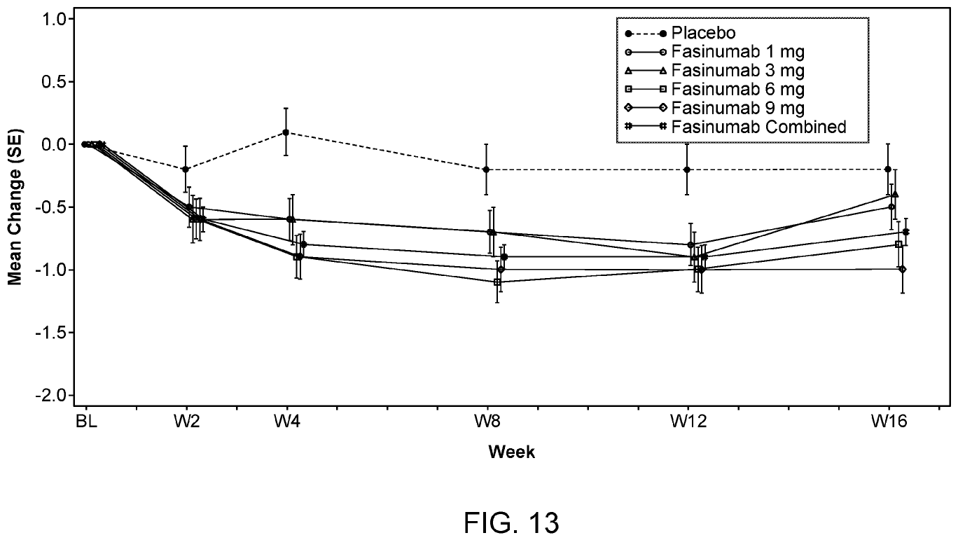

However, subjects in the high centralized pain group who received fasinumab showed an enhanced reduction in EQ-5D-5L item 4 scores when compared to placebo. See FIG. 13 and Tables 14A-14B. As observed in the SF-36 item 7 and 8 results, the reduction in EQ-5D-5L score appeared more pronounced in subjects receiving the 6 mg or 9 mg dose of fasinumab (FIG. 13, open squares and open diamonds, respectively).

TABLE 14A
Change from Baseline in EQ5D-Pain/Discomfort
(Item 4) Score - FM Score 9-31 (mean (SD))

| | Placebo | 1 mg | 3 mg | 6 mg | 9 mg | Combined |
|---|---|---|---|---|---|---|
| Baseline | 3.5 (0.78) | 3.4 (0.68) | 3.2 (0.91) | 3.4 (0.56) | 3.5 (0.71) | 3.4 (0.71) |
| Week 2 | −0.4 (0.88) | −0.6 (1.05) | −0.5 (1.10) | −0.6 (0.92) | −0.7 (1.06) | −0.6 (1.02) |
| Week 4 | −0.1 (0.99) | −0.7 (1.00) | −0.5 (1.17) | −0.9 (0.99) | −1.0 (1.21) | −0.8 (1.09) |
| Week 8 | −0.5 (1.01) | −0.8 (1.06) | −0.5 (1.12) | −1.1 (1.15) | −1.1 (1.20) | −09 (1.14) |
| Week 12 | −0.5 (0.93) | −0.9 (1.24) | −0.7 (1.01) | −1.0 (0.18) | −1.2 (1.31) | −0.9 (1.15) |
| Week 16 | −0.5 (0.96) | −0.7 (1.16) | −0.3 (1.08) | −0.8 (0.99) | −1.2 (1.20) | −0.8 (1.14) |

TABLE 14B
Change from Baseline in EQ5D-Pain/Discomfort
(Item 4) Score - FM Score 9-31 (mean LS (SE))

| | Placebo | 1 mg | 3 mg | 6 mg | 9 mg | Combined |
|---|---|---|---|---|---|---|
| Week 2 | −0.2 (0.18) | −0.5 (0.16) | −0.6 (0.19) | −0.6 (0.16) | −0.6 (0.17) | −0.6 (0.10) |
| Week 4 | 0.1 (0.19) | −0.6 (0.17) | −0.6 (0.20) | −0.9 (0.17) | −0.9 (0.24) | −0.8 (0.19) |
| Week 8 | −0.2 (0.20) | −0.7 (0.17) | −0.7 (0.20) | −1.1 (0.17) | −1.0 (0.18) | −0.9 (0.10) |
| Week 12 | −0.2 (0.20) | −0.8 (0.17) | −0.9 (0.20) | −1.0 (0.18) | −1.0 (0.19) | −0.9 (0.10) |
| Week 16 | −0.2 (0.20) | −0.5 (0.18) | −0.4 (0.20) | −0.8 (0.18) | −1.0 (0.19) | −0.7 (0.11) |

For all three generalized pain scores analyzed in this study, subjects with high levels of centralized pain when treated with fasinumab reported substantial reductions in generalized pain. Therefore it appears that treatment with fasinumab may provide effective pain relief in subjects with high levels of centralized pain prior to treatment.

Conclusion: For all three centralized pain scores analyzed in this study, subjects with high levels of centralized pain when treated with fasinumab reported substantial reductions in generalized pain over time as compared to subjects receiving placebo. Furthermore, fasinumab also appears to be more effective in reducing pain in subjects with higher levels of centralized pain prior to treatment. Since patients reporting higher centralized pain scores have been correlated with poorer pain improvement outcomes (Brummett 2015), fasinumab may represent a novel therapy for reducing pain in this difficult-to-treat patient population.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

TABLE 15
Nucleic acid and amino acid sequences

| SEQ | | |
|---|---|---|
| ID | Descrip- | |
| NO. | tion | SEQUENCE |
| 1. | Fasinumab | CAGGTGCAGC TGGTGCAGTC TGGGGCTGAG GTGAAGAAGC CTGGGGCCTC AGTGAAGGTC |
| | HCVR, | TCCTGCAAGG TTTCCGGATT CACCCTCACT GAATTATCCA TTCACTGGGT GCGACAGGCT |
| | nucleic | CCTGGAAAAG GGCTTGAGTG GATGGGAGGT TTTGATCCTG AAGATGGTGA AACAATCTAC |
| | acid | GCACAGAAGT TCCAGGGCAG AGTCACCATG ACCGAGGACA CATCTACAGA CACAGCCTAC |
| | sequence | ATGGAGCTGA CCAGCCTGAG ATCGGAAGAC ACGGCCGTGT ATTACTGTTC AACGATTTTT |
| | | GGAGTGGTTA CCAACTTTGA CAACTGGGGC CAGGGAACCC TGGTCACCGT CTCCTCA |
| 2. | Fasinumab | QVQLVQSGAE VKKPGASVKV SCKVSGFTLT ELSIHWVRQA PGKGLEWMGG FDPEDGETIY |
| | HCVR, | AQKFQGRVTM TEDTSTDTAY MELTSLRSED TAVYYCSTIF GVVTNFDNWG QGTLVTVSS |
| | amino acid | |
| | sequence | |
| 3. | Fasinumab | GGATTCACCC TCACTGAATT ATCC |
| | HCDR1, | |
| | nucleic | |
| | acid | |
| | sequence | |
| 4. | Fasinumab | GFTLTELS |
| | HCDR1, | |
| | amino acid | |
| | sequence | |
| 5. | Fasinumab | TTTGATCCTG AAGATGGTGA AACA |
| | HCDR2, | |
| | nucleic | |
| | acid | |
| | sequence | |
| 6. | fasinumab | FDPEDGET |
| | HCDR2, | |
| | amino acid | |
| | sequence | |
| 7. | Fasinumab | TCAACGATTT TTGGAGTGGT TACCAACTTT GACAAC |
| | HCDR3, | |
| | nucleic | |
| | acid | |
| | sequence | |
| 8. | fasinumab | STIFGVVTNF DN |
| | HCDR3, | |
| | amino acid | |
| | sequence | |
| 9. | Fasinumab | GACATCCAGA TGACCCAGTC TCCATCCTCC CTGTCTGCAT CTGCAGGAGA CAGAGTCACC |
| | LCVR, | ATCACTTGCC GGGCAAGTCA GGCCATTAGA AATGATTTAG GCTGGTATCA GCAGAAACCA |
| | nucleic | GGGAAAGCCC CTAAGCGCCT GATCTATGCT GCATTCAATT TGCAAAGTGG GGTCCCATCA |
| | acid | AGATTCAGCG GCAGTGGATC TGGGACAGAA TTCACTCTCA CAATCAGTAG CCTGCAGCCT |
| | sequence | GAAGATCTTG CAAGTTATTA CTGTCAACAG TATAATAGAT ACCCGTGGAC GTTCGGCCAA |
| | | GGGACCAAGG TGGAAATCAA ACGA |
| 10. | Fasinumab | DIQMTQSPSS LSASAGDRVT ITCRASQAIR NDLGWYQQKP GKAPKRLIYA AFNLQSGVPS |
| | LCVR, | RFSGSGSGTE FTLTISSLQP EDLASYYCQQ YNRYPWTFGQ GTKVEIKR |
| | amino acid | |
| | sequence | |
| 11. | Fasinumab | CAGGCCATTA GAAATGAT |
| | LCDR1, | |
| | nucleic | |
| | acid | |
| | sequence | |
| 12. | Fasinumab | QAIRND |
| | LCDR1, | |
| | amino acid | |
| | sequence | |
| 13. | Fasinumab | GCTGCATTC |
| | LCDR2, | |
| | nucleic | |
| | acid | |
| | sequence | |
| 14. | Fasinumab | AAF |
| | LCDR2, | |
| | amino acid | |
| | sequence | |
| 15. | Fasinumab | CAACAGTATA ATAGATACCC GTGGACG |
| | LCDR3, | |
| | nucleic | |
| | acid | |
| | sequence | |
| 16. | Fasinumab | QQYNRYPWT |
| | LCDR3, | |
| | amino acid | |
| | sequence | |
| 17. | human | AGCGTCCGGA CCCAATAACA GTTTTACCAA GGGAGCAGCT TTCTATCCTG GCCACACTGA |
| | NGF, | GGTGCATAGC GTAATGTCCA TGTTGTTCTA CACTCTGATC ACAGCTTTTC TGATCGGCAT |
| | nucleic | ACAGGCGGAA CCACACTCAG AGAGCAATGT CCCTGCAGGA CACACCATCC CCCAAGCCCA |
| | acid | CTGGACTAAA CTTCAGCATT CCCTTGACAC TGCCCTTCGC AGAGCCCGCA GCGCCCCGGC |
| | sequence | AGCGGCGATA GCTGCACGCG TGGCGGGGCA GACCCGCAAC ATTACTGTGG ACCCCAGGCT |
| | | GTTTAAAAAG CGGCGACTCC GTTCACCCCG TGTGCTGTTT AGCACCCAGC CTCCCCGTGA |
| | | AGCTGCAGAC ACTCAGGATC TGGACTTCGA GGTCGGTGGT GCTGCCCCCT TCAACAGGAC |
| | | TCACAGGAGC AAGCGGTCAT CATCCCATCC CATCTTCCAC AGGGGCGAAT TCTCGGTGTG |
| | | TGACAGTGTC AGCGTGTGGG TTGGGGATAA GACCACCGCC ACAGACATCA AGGGCAAGGA |
| | | GGTGATGGTG TTGGGAGAGG TGAGCATTAA CAACAGTGTA TTCAAACAGT ACTTTTTTGA |
| | | GACCAAGTGC CGGGACCCAA ATCCCGTTGA CAGCGGGTGC CGGGGCATTG ACTCAAAGCA |
| | | CTGGAACTCA TATTGTACCA CGACTCACAC CTTTGTCAAG GCGCTGACCA TGGATGGCAA |
| | | GCAGGCTGCC TGGCGGTTTA TCCGGATAGA TACGGCCTGT ATGTGTGTGC TCAGCAGGAA |
| | | GGCTGTGAGA AGAGCCTGAC CTGCCGACAC GCTCCCTCCC CCTGCCCCTT CTACACTCTC |
| | | CTGGGCC |
| 18. | human | SSSHPIFHRG EFSVVSVWVG DKTTATDIKG KEVMVLGEVN INNSVFKQYF FETKCRDPNP |
| | NGF, | VDSGCRGIDS KHWNSYCTTT HTFALTMDGK QAAWRFIRID TACVCVLSRK AVRRA |
| | amino acid | |
| | sequence | |

What is claimed is:

1. A method for reducing centralized pain comprising:
(a) selecting a subject with centralized pain; and
(b) administering to the subject a pharmaceutical composition comprising an anti-nerve growth factor (NGF) antibody comprising a heavy chain variable region (HCVR)/light chain variable region (LCVR) amino acid sequence pair of SEQ ID NOs: 2/10.

2. The method of claim 1, wherein the subject suffers from a high level of centralized pain.

3. The method of claim 2, wherein the high level of centralized pain is measured by a Survey of Pain Classification (QSPC) score.

4. The method of claim 3, wherein the QSPC score is the score from the 2011 diagnostic criteria for fibromyalgia from the American College of Rheumatology (FM score).

5. The method of claim 4, wherein the subject has a FM score of 9 or above at baseline.

6. The method of claim 5, wherein the subject has a FM score of 13 or above at baseline.

7. The method of any one of claims 1-6, wherein the efficacy of the NGF antibody in reducing centralized pain is measured by the change from baseline of a QSPC or FM score for the subject.

8. The method of any one of claims 1-7, wherein the efficacy of the NGF antibody in reducing centralized pain is measured by the change from baseline of a generalized pain score for the subject.

9. The method of any one of claims 1-6, wherein the efficacy of the NGF antibody in reducing centralized pain is measured by the change from baseline in a Short Form Health Survey (SF-36) score for the subject.

10. The method of claim 9, wherein the efficacy of the NGF antibody in reducing centralized pain is measured by the change from baseline in the SF-36 item 7 (bodily pain) score for the subject.

11. The method of claim 9, wherein the efficacy of the NGF antibody in reducing centralized pain is measured by the change from baseline in the SF-36 item 8 (pain interference) score for the subject.

12. The method of any one of claims 1-8, wherein the efficacy of the NGF antibody in reducing centralized pain is measured by the change from baseline in a EuroQoL 5 Dimensions 5 Level Questionnaire (EQ-5D-5L) score for the subject.

13. The method of claim 12, wherein the efficacy of the NGF antibody in reducing centralized pain is measured by the change from baseline in an EQ-5D-5L item 4 (pain/discomfort) score for the subject.

14. The method of any one of claims 1-13, wherein the subject is an adult.

15. The method of any one of claims 1-14, wherein the subject suffers from fibromyalgia.

16. The method of any one of claims 1-15, wherein the subject suffers from osteoarthritis.

17. A method for reducing pain or improving physical function, the method comprising:
(a) selecting a patient suffering from osteoarthritis of the knee or hip and centralized pain; and
(b) administering to the subject a pharmaceutical composition comprising an anti-nerve growth factor (NGF) antibody comprising a heavy chain variable region (HCVR)/light chain variable region (LCVR) amino acid sequence pair of SEQ ID NOs: 2/10.

18. The method of claim 17, wherein the subject suffers from a high level of centralized pain.

19. The method of claim 18, wherein the high level of centralized pain is measured by a Survey of Pain Classification (QSPC) score.

20. The method of claim 19, wherein the QSPC score is the score from the 2011 diagnostic criteria for fibromyalgia from the American College of Rheumatology (FM score).

21. The method of claim 20, wherein the subject has a FM score of 9 or above at baseline.

22. The method of claim 21, wherein the subject has a FM score of 13 or above at baseline.

23. The method of any one of claims 17-22, wherein the subject is an adult.

24. The method of any one of claims 17-23, wherein the subject suffers from fibromyalgia.

25. The method of any one of claims 17-24, wherein the efficacy of the NGF antibody in reducing pain or improving physical function is measured by the change from baseline of:
a. the Western Ontario and McMaster Universities Osteoarthritis Index (WOMAC) pain subscale score;
b. the WOMAC physical function subscale score; or
c. the Patient Global Assessment (PGA) score.

26. The method of any one of claims 17-24, wherein the efficacy of the NGF antibody in reducing pain or improving physical function is measured by the change from baseline of:
a. the Numeric Rating Scale of the average walking index joint pain,
b. the EuroQoL 5 Dimensions 5 Level Questionnaire,
c. the 36-item Short Form Survey,
d. the Healthcare Resource Utilization Questionnaire,
e. the Work Productivity and Activity Impairment-Osteoarthritis, or
f. the Treatment Satisfaction Questionnaire for Medication.

27. The method of any one of claims 1-26, wherein the NGF antibody is administered at a dose of about 1 mg every 4 weeks (Q4W).

28. The method of any one of claims 1-26, wherein the NGF antibody is administered at a dose of about 3 mg every 4 weeks (Q4W).

29. The method of any one of claims 1-26, wherein the NGF antibody is administered at a dose of about 6 mg every 4 weeks (Q4W).

30. The method of any one of claims 1-26, wherein the NGF antibody is administered at a dose of about 9 mg every 4 weeks (Q4W).

31. The method of any one of claims 1-26, wherein the NGF antibody is administered at a dose of about 1 mg every 8 weeks (Q8W).

32. The method of any one of claims 1-31, wherein the NGF antibody is fasinumab.
